SEQUENCE LISTING (95 sequences)

SEQ ID NO: 1 - DNA, 1680 bp, Klebsiella pneumoniae
atggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaa

SEQ ID NO: 2 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 3 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 4 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 5 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 6 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 7 - DNA, 1647 bp, Lactococcus lactis
atgtatactgtgggggattacctgctggatcgcctgcacgaactggggattgaagaaattttcggtgtgccaggcgattataacctgcagttcctggaccagattatctcgcacaaagatatgaagtgggtcggtaacgccaacgaactgaacgcgagctatatggcagatggttatgcccgtaccaaaaaagctgctgcgtttctgacgacctttggcgttggcgaactgagcgccgtcaacggactggcaggaagctacgccgagaacctgccagttgtcgaaattgttgggtcgcctacttctaaggttcagaatgaaggcaaatttgtgcaccatactctggctgatggggattttaaacattttatgaaaatgcatgaaccggttactgcggcccgcacgctgctgacagcagagaatgctacggttgagatcgaccgcgtcctgtctgcgctgctgaaagagcgcaagccggtatatatcaatctgcctgtcgatgttgccgcagcgaaagccgaaaagccgtcgctgccactgaaaaaagaaaacagcacctccaatacatcggaccaggaaattctgaataaaatccaggaatcactgaagaatgcgaagaaaccgatcgtcatcaccggacatgagatcatctcttttggcctggaaaaaacggtcacgcagttcatttctaagaccaaactgcctatcaccaccctgaacttcggcaaatctagcgtcgatgaagcgctgccgagttttctgggtatctataatggtaccctgtccgaaccgaacctgaaagaattcgtcgaaagcgcggactttatcctgatgctgggcgtgaaactgacggatagctccacaggcgcatttacccaccatctgaacgagaataaaatgatttccctgaatatcgacgaaggcaaaatctttaacgagcgcatccagaacttcgattttgaatctctgattagttcgctgctggatctgtccgaaattgagtataaaggtaaatatattgataaaaaacaggaggattttgtgccgtctaatgcgctgctgagtcaggatcgtctgtggcaagccgtagaaaacctgacacagtctaatgaaacgattgttgcggaacagggaacttcatttttcggcgcctcatccatttttctgaaatccaaaagccatttcattggccaaccgctgtgggggagtattggttatacctttccggcggcgctgggttcacagattgcagataaggaatcacgccatctgctgtttattggtgacggcagcctgcagctgactgtccaggaactggggctggcgatccgtgaaaaaatcaatccgatttgctttatcatcaataacgacggctacaccgtcgaacgcgaaattcatggaccgaatcaaagttacaatgacatcccgatgtggaactatagcaaactgccggaatcctttggcgcgacagaggatcgcgtggtgagtaaaattgtgcgtacggaaaacgaatttgtgtcggttatgaaagaagcgcaggctgacccgaatcgcatgtattggattgaactgatcctggcaaaagaaggcgcaccgaaagttctgaaaaagatggggaaactgtttgcggagcaaaataaaagctaa

SEQ ID NO: 8 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 9 - DNA, 1047 bp, Achromobacter xyloxidans
atgaaagctctggtttatcacggtgaccacaagatctcgcttgaagacaagcccaagcccacccttcaaaagcccacggatgtagtagtacgggttttgaagaccacgatctgcggcacggatctcggcatctacaaaggcaagaatccagaggtcgccgacgggcgcatcctgggccatgaaggggtaggcgtcatcgaggaagtgggcgagagtgtcacgcagttcaagaaaggcgacaaggtcctgatttcctgcgtcacttcttgcggctcgtgcgactactgcaagaagcagctttactcccattgccgcgacggcgggtggatcctgggttacatgatcgatggcgtgcaggccgaatacgtccgcatcccgcatgccgacaacagcctctacaagatcccccagacaattgacgacgaaatcgccgtcctgctgagcgacatcctgcccaccggccacgaaatcggcgtccagtatgggaatgtccagccgggcgatgcggtggctattgtcggcgcgggccccgtcggcatgtccgtactgttgaccgcccagttctactccccctcgaccatcatcgtgatcgacatggacgagaatcgcctccagctcgccaaggagctcggggcaacgcacaccatcaactccggcacggagaacgttgtcgaagccgtgcataggattgcggcagagggagtcgatgttgcgatcgaggcggtgggcataccggcgacttgggacatctgccaggagatcgtcaagcccggcgcgcacatcgccaacgtcggcgtgcatggcgtcaaggttgacttcgagattcagaagctctggatcaagaacctgacgatcaccacgggactggtgaacacgaacacgacgcccatgctgatgaaggtcgcctcgaccgacaagcttccgttgaagaagatgattacccatcgcttcgagctggccgagatcgagcacgcctatcaggtattcctcaatggcgccaaggagaaggcgatgaagatcatcctctcgaacgcaggcgctgcctga

SEQ ID NO: 10 - protein, 348 aa, Achromobacter xyloxidans
MKALVYHGDHKISLEDKPKPTLQKPTDVVVRVLKTTICGTDLGIYKGKNPEVADGRILGHEGVGVIEEVGESVTQFKKGDKVLISCVTSCGSCDYCKKQLYSHCRDGGWILGYMIDGVQAEYVRIPHADNSLYKIPQTIDDEIAVLLSDILPTGHEIGVQYGNVQPGDAVAIVGAGPVGMSVLLTAQFYSPSTIIVIDMDENRLQLAKELGATHTINSGTENVVEAVHRIAAEGVDVAIEAVGIPATWDICQEIVKPGAHIANVGVHGVKVDFEIQKLWIKNLTITTGLVNTNTTPMLMKVASTDKLPLKKMITHRFELAEIEHAYQVFLNGAKEKAMKIILSNAGAA

SEQ ID NO: 11 - DNA, 1713 bp, Bacillus subtilis
ttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 12 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 13 - DNA, 1188 bp, Saccharomyces cerevisiae
atgttgagaactcaagccgccagattgatctgcaactcccgtgtcatcactgctaagagaacctttgctttggccacccgtgctgctgcttacagcagaccagctgcccgtttcgttaagccaatgatcactacccgtggtttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataa

SEQ ID NO: 14 - protein, 395 aa, Saccharomyces cerevisiae
MLRTQAARLICNSRVITAKRTFALATRAAAYSRPAARFVKPMITTRGLKQINFGGTVETVYERADWPREKLLDYFKNDTFALIGYGSQGYGQGLNLRDNGLNVIIGVRKDGASWKAAIEDGWVPGKNLFTVEDAIKRGSYVMNLLSDAAQSETWPAIKPLLTKGKTLYFSHGFSPVFKDLTHVEPPKDLDVILVAPKGSGRTVRSLFKEGRGINSSYAVWNDVTGKAHEKAQALAVAIGSGYVYQTTFEREVNSDLYGERGCLMGGIHGMFLAQYDVLRENGHSPSEAFNETVEEATQSLYPLIGKYGMDYMYDACSTTARRGALDWYPIFKNALKPVFQDLYESTKNGTETKRSLEFNSQPDYREKLEKELDTIRNMEIWKVGKEVRKLRPENQ

SEQ ID NO: 15 - DNA, 1014 bp, artificial sequence
  Mutant of Pseudomonas fluorescens ilvC coding region
atgaaggtgttttacgataaagactgcgatctgagcatcatccagggaaagaaggttgctattataggatatggttcccaaggacacgcacaagccttgaacttgaaagattctggggtcgacgtgacagtaggtctgtataaaggtgctgctgatgcagcaaaggctgaagcacatggctttaaagtcacagatgttgcagcggctgttgctggcgctgatttagtcatgattttaattccagatgaatttcaatcgcaattgtacaaaaatgaaatagaaccaaacattaagaagggcgctaccttggccttcagtcatggatttgccattcattacaatcaagtagtccccagggcagatttggacgttattatgattgcacctaaggctccggggcatactgttaggagcgaatttgttaagggtggtggtattccagatttgatcgctatataccaagacgttagcggaaacgctaagaatgtagctttaagctacgcagcaggagttggtggcgggagaacgggtataatagaaaccacttttaaagacgagactgagacagatttatttggagaacaagcggttctgtgcggaggaactgttgaattggttaaagcaggctttgagacgcttgtcgaagcagggtacgctcccgaaatggcatacttcgaatgtctacatgaattgaagttgatagtagacttaatgtatgaaggtggtatagctaatatgaactattccatttcaaataatgcagaatatggtgagtatgtcaccggacctgaagtcattaacgcagaatcaagacaagccatgagaaatgccttgaaacgtatccaggacggtgaatacgctaagatgttcataagtgaaggcgctacgggttacccgagtatgactgctaaaagaagaaacaatgcagcacatggtatcgaaattattggtgaacagttaaggtctatgatgccctggatcggtgctaataagatcgtagacaaggcgaaaaat

SEQ ID NO: 16 - protein, 338 aa, artificial sequence
  mutant of Pseudomonas fluorescens protein
MKVFYDKDCDLSIIQGKKVAIIGYGSQGHAQALNLKDSGVDVTVGLYKGAADAAKAEAHGFKVTDVAAAVAGADLVMILIPDEFQSQLYKNEIEPNIKKGATLAFSHGFAIHYNQVVPRADLDVIMIAPKAPGHTVRSEFVKGGGIPDLIAIYQDVSGNAKNVALSYAAAVGGGRTGIIETTFKDETETDLFGEQAVLCGGTVELVKAGFETLVEAGYAPEMAYFECLHELKLIVDLMYEGGIANMNYSISNNAEYGEYVTGPEVINAESRQAMRNALKRIQDGEYAKMFISEGATGYPSMTAKRRNNAAHGIEIIGEQLRSMMPWIGANKIVDKAKN

SEQ ID NO: 17 - DNA, 1713 bp, Streptococcus mutans
atgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaa

SEQ ID NO: 18 - protein, 571 aa, Streptococcus mutans
MTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIILHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGILGKYAHIVSSASRGAVTDFWKPEETGKK

SEQ ID NO: 19 - DNA, 1644 bp, artificial sequence
  Bacillus subtilis kivD coding region codon optimized for expression is S. cerevisiae
atgtatacagtaggtgactatctgttggacagattacacgaattaggtatagaagaaatattcggagtaccaggtgactacaatttgcaatttctagatcaaattatttcacacaaagatatgaaatgggtgggaaatgctaatgagttaaatgcctcctatatggccgacgggtacgcaagaacgaaaaaggctgcggcattcttgactacatttggtgttggcgaattatccgcagttaatggcttagcgggctcctatgctgagaacctgcctgttgttgagatcgtgggatctcctacctcgaaagtgcagaacgaaggtaagtttgttcaccatacgttggctgatggtgatttcaagcactttatgaagatgcacgaaccggttactgctgccaggactttattgacagccgagaatgcaactgttgaaattgatagagtgttgtctgccttactaaaggaaagaaagccggtttacatcaatttacctgtagatgtagctgccgctaaggctgaaaaaccatccttgcctcttaagaaggaaaattccacgtcgaatacatctgatcaagagattctgaacaaaatacaggaaagtctgaagaatgccaagaaaccaattgtaatcacaggccatgaaattatatcgttcggcctagagaagactgttactcagtttatttcaaagactaagttacctattactactttgaactttggtaaatcatctgttgatgaagcattgccctcatttttggggatttacaacggtactctgtcagagccaaacttgaaggaatttgtggaatctgctgattttattcttatgttgggtgtaaagcttaccgattctagtacgggtgcatttactcaccatcttaatgaaaataaaatgatttccttgaatatcgatgaaggtaaaattttcaacgaaagaatccaaaatttcgacttcgaatccctgatatcatctcttcttgacttgtccgaaattgaatataaaggcaagtacatagataaaaagcaagaagattttgtaccttctaacgcgctgttgtcacaagatagactgtggcaagctgtcgaaaatttgacccaaagtaatgagacgatcgtggctgaacaaggcacttctttcttcggtgcctcatctatatttctgaaatcgaaatcacattttattggtcaacccttgtggggatctataggatacactttccccgcagctctaggcagccaaattgcagataaagaatctagacatttattgtttatcggagatggatcattgcaactgactgtccaagaattaggactagccattagagagaagataaacccaatctgctttatcattaataacgatggttacacggttgagagggaaattcatggtccgaaccagagttataatgacattcctatgtggaattactcaaaactgccagaaagtttcggggcaacggaagacagagttgtgtccaaaattgtgagaacagaaaatgaattcgtatccgtgatgaaagaagctcaagcagatccaaataggatgtattggatagaacttattctagcaaaggagggtgcacctaaagttttgaaaaagatgggtaagttatttgcagaacaaaacaagagc

SEQ ID NO: 20 - protein, 548 aa, Bacillus subtilis; identical to SEQ ID NO: 8

SEQ ID NO: 21 - DNA, 2145 bp, artificial sequence
  constructed chimeric gene
gcatgcttgcatttagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaatagctagctgaggatgaaggcattagtttatcatggggatcacaaaatttcgttagaagacaaaccaaaacccactctgcagaaaccaacagacgttgtggttagggtgttgaaaacaacaatttgcggtactgacttgggaatatacaaaggtaagaatcctgaagtggcagatggcagaatcctgggtcatgagggcgttggcgtcattgaagaagtgggcgaatccgtgacacaattcaaaaagggggataaagttttaatctcctgcgttactagctgtggatcgtgtgattattgcaagaagcaactgtattcacactgtagagacggtggctggattttaggttacatgatcgacggtgtccaagccgaatacgtcagaataccacatgctgacaattcattgtataagatcccgcaaactatcgatgatgaaattgcagtactactgtccgatattttacctactggacatgaaattggtgttcaatatggtaacgttcaaccaggcgatgctgtagcaattgtaggagcaggtcctgttggaatgtcagttttgttaactgctcaattttactcgcctagtaccattattgttatcgacatggacgaaaaccgtttacaattagcgaaggagcttggggccacacacactattaactccggtactgaaaatgttgtcgaagctgtgcatcgtatagcagccgaaggagtggatgtagcaatagaagctgttggtatacccgcaacctgggacatctgtcaggaaattgtaaaacccggcgctcatattgccaacgtgggagttcatggtgttaaggtggactttgaaattcaaaagttgtggattaagaatctaaccatcaccactggtttggttaacactaatactaccccaatgttgatgaaggtagcctctactgataaattgcctttaaagaaaatgattactcacaggtttgagttagctgaaatcgaacacgcatatcaggttttcttgaatggcgctaaagaaaaagctatgaagattattctatctaatgcaggtgccgcctaattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggt

SEQ ID NO: 22 - DNA, 4280 bp, artificial sequence
  vector
ggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccccggctctgagacagtagtaggttagtcatcgctctaccgacgcgcaggaaaagaaagaagcattgcggattacgtattctaatgttcagcccgcggaacgccagcaaatcaccacccatgcgcatgatactgagtcttgtacacgctgggcttccagtgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcttcagcccgcggaacgccagcaaatcaccacccatgcgcatgatactgagtcttgtacacgctgggcttccagtgatgatacaacgagttagccaaggtgagcacggatgtctaaattagaattacgttttaatatctttttttccatatctagggctag

SEQ ID NO: 23 - DNA, 30 bp, artificial sequence
  primer
gcatgcttgcatttagtcgtgcaatgtatg

SEQ ID NO: 24 - DNA, 54 bp, artificial sequence
  primer
gaacattagaatacgtaatccgcaatgcactagtaccacaggtgttgtcctctg

SEQ ID NO: 25 - DNA, 54 bp, artificial sequence
  primer
cagaggacaacacctgtggtactagtgcattgcggattacgtattctaatgttc

SEQ ID NO: 26 - DNA, 28 bp, artificial sequence
  primer
caccttggctaactcgttgtatcatcac

SEQ ID NO: 27 - DNA, 100 bp, artificial sequence
  primer
ttttaagccgaatgagtgacagaaaaagcccacaacttatcaagtgatattgaacaaagggcgaaacttcgcatgcttgcatttagtcgtgcaatgtatg

SEQ ID NO: 28 - DNA, 98 bp, artificial sequence
  primer
cccaattggtaaatattcaacaagagacgcgcagtacgtaacatgcgaattgcgtaattcacggcgataacaccttggctaactcgttgtatcatcac

SEQ ID NO: 29 - DNA, 28 bp, artificial sequence
  primer
tcggtttttgcaatatgacctgtgggcc

SEQ ID NO: 30 - DNA, 29 bp, artificial sequence
  primer
caaaagcccatgtcccacaccaaaggatg

SEQ ID NO: 31 - DNA, 26 bp, artificial sequence
  primer
caccatcgcgcgtgcatcactgcatg

SEQ ID NO: 32 - DNA, 22 bp, artificial sequence
  primer
gagaagatgcggccagcaaaac

SEQ ID NO: 33 - DNA, 2745 bp, artificial sequence
  constructed coding region-terminator segment
atgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaa

SEQ ID NO: 34 - DNA, 99 bp, artificial sequence
  primer
tcctttctcaattattattttctactcataacctcacgcaaaataacacagtcaaatcaatcaaagtatgactgacaaaaaaactcttaaagacttaag

SEQ ID NO: 35 - DNA, 77 bp, artificial sequence
  primer
gaacattagaatacgtaatccgcaatgcttctttcttttccgtttaacgtatagacttctaatatatttctccatac

SEQ ID NO: 36 - DNA, 45 bp, artificial sequence
  primer
aaacggaaaagaaagaagcattgcggattacgtattctaatgttc

SEQ ID NO: 37 - DNA, 88 bp, artificial sequence
  primer
tatttttcgttacataaaaatgcttataaaactttaactaataattagagattaaatcgccaccttggctaactcgttgtatcatcac

SEQ ID NO: 38 - DNA, 27 bp, artificial sequence
  primer
gacttttggaagcctgaagaaactggc

SEQ ID NO: 39 - DNA, 20 bp, artificial sequence
  primer
cttggcagcaacaggactag

SEQ ID NO: 40 - DNA, 26 bp, artificial sequence
  primer
ccaggccaattcaacagactgtcggc

SEQ ID NO: 41 - DNA, 2347 bp, artificial sequence
  constructed URA3 marker with flanking homologous repeat sequences for HIS gene replacement and marker excision
gcattgcggattacgtattctaatgttcaggtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatggtacctactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatctctagagtgctggaagaagagctgcttaaccgccgcgcccagggtgaagatccacgctactttaccctgcgtcgtctggatttcggcggctgtcgtctttcgctggcaacgccggttgatgaagcctgggacggtccgctctccttaaacggtaaacgtatcgccacctcttatcctcacctgctcaagcgttatctcgaccagaaaggcatctcttttaaatcctgcttactgaacggttctgttgaagtcgccccgcgtgccggactggcggatgcgatttgcgatctggtttccaccggtgccacgctggaagctaacggcctgcgcgaagtcgaagttatctatcgctcgaaagcctgcctgattcaacgcgatggcgaaatggaagaatccaaacagcaactgatcgacaaactgctgacccgtattcagggtgtgatccaggcgcgcgaatcaaaatacatcatgatgcacgcaccgaccgaacgtctggatgaagtcatccagtgatgatacaacgagttagccaaggtg

SEQ ID NO: 42 - DNA, 80 bp, artificial sequence
  primer
cttcgaagaatatactaaaaaatgagcaggcaagataaacgaaggcaaaggcattgcggattacgtattctaatgttcag

SEQ ID NO: 43 - DNA, 80 bp, artificial sequence; identical to SEQ ID NO: 42
  primer

SEQ ID NO: 44 - DNA, 26 bp, artificial sequence
  primer
gacttgaataatgcagcggcgcttgc

SEQ ID NO: 45 - DNA, 30 bp, artificial sequence
  primer
ccaccctcttcaattagctaagatcatagc

SEQ ID NO: 46 - DNA, 25 bp, artificial sequence
  primer
aaaaattgattctcatcgtaaatgc

SEQ ID NO: 47 - DNA, 20 bp, artificial sequence
  primer
ctgcagcgaggagccgtaat

SEQ ID NO: 48 - DNA, 16387 bp, artificial sequence
  plasmid construct
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcgctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaagcatctaaaacacaaccgttggaagcgttggaaaccaacttagcatacttggatagagtacctcttgtgtaacgaggtggaggtgcaacccaactttgtttacgttgagccatttccttatcagagactaataggtcaatcttgttattatcagcatcaatgataatctcatcgccgtctctgaccaacccgataggaccaccttcagcggcttcgggaacaatgtggccgattaagaacccgtgagaaccaccagagaatctaccatcagtcaacaatgcaacatctttacccaaaccgtaacccatcagagcagaggaaggctttagcatttcaggcatacctggtgcacctcttggaccttcatatctgataacaacaacggttttttcacccttcttgatttcacctctttccaaggcttcaataaaggcaccttcctcttcgaacacacgtgctctacccttgaagtaagtaccttccttaccggtaattttacccacagctccacctggtgccaatgaaccgtacagaatttgcaagtgaccgttggccttgattgggtgggagagtggcttaataatctcttgtccttcaggtaggcttggtgctttctttgcacgttctgccaaagtgtcaccggtaacagtcattgtgttaccgtgcaacatgttgttttcatatagatacttaatcacagattgggtaccaccaacgttaatcaaatcggccatgacgtatttaccagaaggtttgaagtcaccgatcaatggtgtagtatcactgattctttggaaatcatctggtgacaacttgacacccgcagagtgagcaacagccaccaaatgcaaaacagcattagtggacccaccggttgcaacgacataagtaatggcgttttcaaaagcctcttttgtgaggatatcacgaggtaaaatacccaattccattgtcttcttgatgtattcaccaatgttgtcacactcagctaacttctccttggaaacggctgggaaggaagaggagtttggaatggtcaaacctagcacttcagcggcagaagccattgtgttggcagtatacataccaccacaagaaccaggacctgggcatgcatgttccacaacatcttctctttcttcttcagtgaattgcttggaaatatattcaccgtaggattggaacgcagagacgatatcgatgtttttagagatcctgttaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactagtatgaaggtgttttacgataaagactgcgatctgagcatcatccagggaaagaaggttgctattataggatatggttcccaaggacacgcacaagccttgaacttgaaagattctggggtcgacgtgacagtaggtctgtataaaggtgctgctgatgcagcaaaggctgaagcacatggctttaaagtcacagatgttgcagcggctgttgctggcgctgatttagtcatgattttaattccagatgaatttcaatcgcaattgtacaaaaatgaaatagaaccaaacattaagaagggcgctaccttggccttcagtcatggatttgccattcattacaatcaagtagtccccagggcagatttggacgttattatgattgcacctaaggctccggggcatactgttaggagcgaatttgttaagggtggtggtattccagatttgatcgctatataccaagacgttagcggaaacgctaagaatgtagctttaagctacgcagcaggagttggtggcgggagaacgggtataatagaaaccacttttaaagacgagactgagacagatttatttggagaacaagcggttctgtgcggaggaactgttgaattggttaaagcaggctttgagacgcttgtcgaagcagggtacgctcccgaaatggcatacttcgaatgtctacatgaattgaagttgatagtagacttaatgtatgaaggtggtatagctaatatgaactattccatttcaaataatgcagaatatggtgagtatgtcaccggacctgaagtcattaacgcagaatcaagacaagccatgagaaatgccttgaaacgtatccaggacggtgaatacgctaagatgttcataagtgaaggcgctacgggttacccgagtatgactgctaaaagaagaaacaatgcagcacatggtatcgaaattattggtgaacagttaaggtctatgatgccctggatcggtgctaataagatcgtagacaaggcgaaaaattaaggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagtatataatttttcaggtaaaatttagtacgatagtaaaatacttctcgaactcgtcacatatacgtgtacataatgtctgaaccagctcaaaagaaacaaaaggttgctaacaactctctagagcggccgcccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgattaattaattattggttttctggtctcaactttctgacttccttaccaaccttccagatttccatgtttctgatggtgtctaattccttttctagcttttctctgtagtcaggttgagagttgaattccaaagatctcttggtttcggtaccgttcttggtagattcgtacaagtcttggaaaacaggcttcaaagcattcttgaagattgggtaccagtccaaagcacctcttctggcggtggtggaacaagcatcgtacatgtaatccataccgtacttaccgatcaatgggtatagagattgggtagcttcttcgacggtttcgttgaaagcttcagatggggagtgaccgttttctctcaagacgtcgtattgagccaagaacataccgtggataccacccattaaacaacctctttcaccgtacaagtcagagttgacttctctttcgaaagtggtttggtaaacgtaaccggaaccaatggcaacggccaaagcttgggccttttcgtgagccttaccggtgacatcgttccagacggcgtaagaagagttaataccacgaccttccttgaacaaagatctgacagttctaccggaaccctttggagcaaccaagataacatctaagtcctttggtggttcaacgtgagtcaagtccttgaagactggggagaaaccgtgggagaagtacaaagtcttacccttggtcaacaatggcttgatagcaggccaggtttctgattgagcggcatcggacaacaagttcataacgtaactacctctcttgatagcatcttcaacagtgaacaagttcttgcctggaacccaaccgtcttcgatggcagccttccaagaagcaccatctttacggacaccaatgataacgttcaaaccgttgtctctcaagttcaaaccttgaccgtaaccttgggaaccgtaaccgatcaaagcaaaagtgtcgttcttgaagtagtccaacaacttttctcttggccagtcagctctttcgtagacggtttcaacagtaccaccgaagttgatttgcttcaacatcctcagctctagatttgaatatgtattacttggttatggttatatatgacaaaagaaaaagaagaacagaagaataacgcaaggaagaacaataactgaaattgatagagaagtattatgtctttgtctttttataataaatcaagtgcagaaatccgttagacaacatgagggataaaatttaacgtgggcgaagaagaaggaaaaaagtttttgtgagggcgtaattgaagcgatctgttgattgtagattttttttttttgaggagtcaaagtcagaagagaacagacaaatggtattaaccatccaatacttttttggagcaacgctaagctcatgcttttccattggttacgtgctcagttgttagatatggaaagagaggatgctcacggcagcgtgactccaattgagcccgaaagagaggatgccacgttttcccgacggctgctagaatggaaaaaggaaaaatagaagaatcccattcctatcattatttacgtaatgacccacacatttttgagattttcaactattacgtattacgataatcctgctgtcattatcattattatctatatcgacgtatgcaacgtatgtgaagccaagtaggcaattatttagtactgtcagtattgttattcatttcagatctatccgcggtggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 49 - DNA, 448 bp, Saccharomyces cerevisiae
cccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatc

SEQ ID NO: 50 - DNA, 250 bp, Saccharomyces cerevisiae
ccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatga

SEQ ID NO: 51 - DNA, 1181 bp, Saccharomyces cerevisiae
taaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactagt

SEQ ID NO: 52 - DNA, 759 bp, Saccharomyces cerevisiae
ggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagtatataatttttcaggtaaaatttagtacgatagtaaaatacttctcgaactcgtcacatatacgtgtacataatgtctgaaccagctcaaaagaaacaaaaggttgctaacaactctctaga

SEQ ID NO: 53 - DNA, 643 bp, Saccharomyces cerevisiae
gaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaatctaga

SEQ ID NO: 54 - DNA, 270 bp, Saccharomyces cerevisiae
gacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaa

SEQ ID NO: 55 - DNA, 15539 bp, artificial sequence
  constructed plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggcctcagctcttgttttgttctgcaaataacttacccatctttttcaaaactttaggtgcaccctcctttgctagaataagttctatccaatacatcctatttggatctgcttgagcttctttcatcacggatacgaattcattttctgttctcacaattttggacacaactctgtcttccgttgccccgaaactttctggcagttttgagtaattccacataggaatgtcattataactctggttcggaccatgaatttccctctcaaccgtgtaaccatcgttattaatgataaagcagattgggtttatcttctctctaatggctagtcctaattcttggacagtcagttgcaatgatccatctccgataaacaataaatgtctagattctttatctgcaatttggctgcctagagctgcggggaaagtgtatcctatagatccccacaagggttgaccaataaaatgtgatttcgatttcagaaatatagatgaggcaccgaagaaagaagtgccttgttcagccacgatcgtctcattactttgggtcaaattttcgacagcttgccacagtctatcttgtgacaacagcgcgttagaaggtacaaaatcttcttgctttttatctatgtacttgcctttatattcaatttcggacaagtcaagaagagatgatatcagggattcgaagtcgaaattttggattctttcgttgaaaattttaccttcatcgatattcaaggaaatcattttattttcattaagatggtgagtaaatgcacccgtactagaatcggtaagctttacacccaacataagaataaaatcagcagattccacaaattccttcaagtttggctctgacagagtaccgttgtaaatccccaaaaatgagggcaatgcttcatcaacagatgatttaccaaagttcaaagtagtaataggtaacttagtctttgaaataaactgagtaacagtcttctctaggccgaacgatataatttcatggcctgtgattacaattggtttcttggcattcttcagactttcctgtattttgttcagaatctcttgatcagatgtattcgacgtggaattttccttcttaagaggcaaggatggtttttcagccttagcggcagctacatctacaggtaaattgatgtaaaccggctttctttcctttagtaaggcagacaacactctatcaatttcaacagttgcattctcggctgtcaataaagtcctggcagcagtaaccggttcgtgcatcttcataaagtgcttgaaatcaccatcagccaacgtatggtgaacaaacttaccttcgttctgcactttcgaggtaggagatcccacgatctcaacaacaggcaggttctcagcataggagcccgctaagccattaactgcggataattcgccaacaccaaatgtagtcaagaatgccgcagcctttttcgttcttgcgtacccgtcggccatataggaggcatttaactcattagcatttcccacccatttcatatctttgtgtgaaataatttgatctagaaattgcaaattgtagtcacctggtactccgaatatttcttctatacctaattcgtgtaatctgtccaacagatagtcacctactgtatacattttgtttactagtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgaaaaagcgtgttttttattcaaaatgattctaactcccttacgtaatcaaggaatctttttgccttggcctccgcgtcattaaacttcttgttgttgacgctaacattcaacgctagtatatattcgtttttttcaggtaagttcttttcaacgggtcttactgatgaggcagtcgcgtctgaacctgttaagaggtcaaatatgtcttcttgaccgtacgtgtcttgcatgttattagctttgggaatttgcatcaagtcataggaaaatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 56 - DNA, 1125 bp, artificial sequence
  horse ADH coding region codon optimized for S. cerevisiae expression
atgtcaacagccggtaaagttattaagtgtaaagcggcagttttgtgggaagagaaaaagccgtttagcatagaagaagtagaagtagcgccaccaaaagcacacgaggttagaatcaagatggttgccaccggaatctgtagatccgacgaccatgtggtgagtggcactctagttactcctttgccagtaatcgcgggacacgaggctgccggaatcgttgaatccataggtgaaggtgttaccactgttcgtcctggtgataaagtgatcccactgttcactcctcaatgtggtaagtgtagagtctgcaaacatcctgagggtaatttctgccttaaaaatgatttgtctatgcctagaggtactatgcaggatggtacaagcagatttacatgcagagggaaacctatacaccatttccttggtacttctacattttcccaatacacagtggtggacgagatatctgtcgctaaaatcgatgcagcttcaccactggaaaaagtttgcttgatagggtgcggattttccaccggttacggttccgcagttaaagttgcaaaggttacacagggttcgacttgtgcagtattcggtttaggaggagtaggactaagcgttattatggggtgtaaagctgcaggcgcagcgaggattataggtgtagacatcaataaggacaaatttgcaaaagctaaggaggtcggggctactgaatgtgttaaccctcaagattataagaaaccaatacaagaagtccttactgaaatgtcaaacggtggagttgatttctcttttgaagttataggccgtcttgatactatggtaactgcgttgtcctgctgtcaagaggcatatggagtcagtgtgatcgtaggtgttcctcctgattcacaaaatttgtcgatgaatcctatgctgttgctaagcggtcgtacatggaagggagctatatttggcggttttaagagcaaggatagtgttccaaaacttgttgccgactttatggcgaagaagtttgctcttgatcctttaattacacatgtattgccattcgagaaaatcaatgaagggtttgatttgttaagaagtggtgaatctattcgtacaattttaactttt

SEQ ID NO: 57 - protein, 375 aa, Equus caballus
MSTAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 58 - DNA, 9089 bp, artificial sequence
  constructed plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggccctaacctgctaggacacaacgtctttgcctggtaaagtttctagctgacgtgattccttcacctgtggatccggcaattgtaaaggttgtgaaaccctcagcttcataaccgacacctgcaaatgactttgcattcttaacaaagatagttgtatcaatttcacgttcgaatctattaaggttatcgatgttcttagaataaatgtaggcggaatgttttctattctgctcagctatcttggcgtatttaatggcttcatcaatgtccttcactctaactataggcaaaattggcatcatcaactccgtcataacgaacggatggtttgcgttgacttcacaaataatacactttacattacttggtgactctacatctatttcatccaaaaacagtttagcgtccttaccaacccacttcttattaatgaaatattcttgagtttcattgttcttttgaagaacaaggtctatcagcttggatacttggtcttcattgataatgacggcgttgtttttcaacatgttagagatcagatcatctgcaacgttttcaaacacgaacacttctttttccgcgatacaaggaagattgttgtcaaacgaacaaccttcaataatgcttctgccggccttctcgatatctgctgtatcgtctacaataaccggaggattacccgcgccagctccgatggcctttttaccagaattaagaagggtttttaccatacccgggccacccgtaccgcacaacaattttatggatggatgtttgataatagcgtctaaactttccatagttgggttctttatagtagtgacaaggttttcaggtccaccacagctaattatggctttgtttatcatttctactgcgaaagcgacacactttttggcgcatgggtgaccattaaatacaactgcattccccgcagctatcatacctatagaattgcagataacggtttctgttggattcgtgcttggagttatagcgccgataactccgtatggactcatttcaaccactgttagtccattatcgccggaccatgctgttgttgtcagatcttcagtgcctggggtatacttggccactaattcatgtttcaagattttatcctcataccttcccatgtgggtttcctccaggatcattgtggctaagacctctttattctgtaatgcggcttttcttatttcggtgattattttctctctttgttcctttgtgtagtgtagggaaagaatcttttgtgcatgtactgcagaagaaatggcattctcaacattttcaaatactccaaaacatgaagagttatctttgtaattctttaagttgatgttttcaccattagtcttcactttcaagtctttggtggttgggattaaggtatctttatccatggtgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgaaaaagcgtgttttttattcaaaatgattctaactcccttacgtaatcaaggaatctttttgccttggcctccgcgtcattaaacttcttgttgttgacgctaacattcaacgctagtatatattcgtttttttcaggtaagttcttttcaacgggtcttactgatgaggcagtcgcgtctgaacctgttaagaggtcaaatatgtcttcttgaccgtacgtgtcttgcatgttattagctttgggaatttgcatcaagtcataggaaaatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 59 - DNA, 672 bp, Saccharomyces cerevisiae
agttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaac

SEQ ID NO: 60 - DNA, 1023 bp, Saccharomyces cerevisiae
caccgcggtggggcgcgccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaactagtaaacaaa

SEQ ID NO: 61 - DNA, 21 bp, artificial sequence
  primer
caaaagctgagctccaccgcg

SEQ ID NO: 62 - DNA, 44 bp, artificial sequence
  primer
gtttactagtttatgtgtgtttattcgaaactaagttcttggtg

SEQ ID NO: 63 - DNA, 8994 bp, artificial sequence
  constructed plasmid
ctagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccgcatgcttgcatttagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaatagctagctgaggatgaaggcattagtttatcatggggatcacaaaatttcgttagaagacaaaccaaaacccactctgcagaaaccaacagacgttgtggttagggtgttgaaaacaacaatttgcggtactgacttgggaatatacaaaggtaagaatcctgaagtggcagatggcagaatcctgggtcatgagggcgttggcgtcattgaagaagtgggcgaatccgtgacacaattcaaaaagggggataaagttttaatctcctgcgttactagctgtggatcgtgtgattattgcaagaagcaactgtattcacactgtagagacggtggctggattttaggttacatgatcgacggtgtccaagccgaatacgtcagaataccacatgctgacaattcattgtataagatcccgcaaactatcgatgatgaaattgcagtactactgtccgatattttacctactggacatgaaattggtgttcaatatggtaacgttcaaccaggcgatgctgtagcaattgtaggagcaggtcctgttggaatgtcagttttgttaactgctcaattttactcgcctagtaccattattgttatcgacatggacgaaaaccgtttacaattagcgaaggagcttggggccacacacactattaactccggtactgaaaatgttgtcgaagctgtgcatcgtatagcagccgaaggagtggatgtagcaatagaagctgttggtatacccgcaacctgggacatctgtcaggaaattgtaaaacccggcgctcatattgccaacgtgggagttcatggtgttaaggtggactttgaaattcaaaagttgtggattaagaatctaaccatcaccactggtttggttaacactaatactaccccaatgttgatgaaggtagcctctactgataaattgcctttaaagaaaatgattactcacaggtttgagttagctgaaatcgaacacgcatatcaggttttcttgaatggcgctaaagaaaaagctatgaagattattctatctaatgcaggtgccgcctaattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggta

SEQ ID NO: 64 - DNA, 753 bp, Saccharomyces cerevisiae
gcatgcttgcatttagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaata

SEQ ID NO: 65 - DNA, 316 bp, Saccharomyces cerevisiae
gagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggt

SEQ ID NO: 66 - DNA, 39 bp, artificial sequence
  primer
cacacatattacaatagctagctgaggatgaaagctctg

SEQ ID NO: 67 - DNA, 39 bp, artificial sequence
  primer
cagagctttcatcctcagctagctattgtaatatgtgtg

SEQ ID NO: 68 - DNA, 9491 bp, artificial sequence
  constructed plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 69 - DNA, 1000 bp, Saccharymoces cerevisiae
gttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaa

SEQ ID NO: 70 - protein, 760 aa, Escherichia coli
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 71 - DNA, 2283 bp, Escherichia coli
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 72 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 73 - DNA, 735 bp, Escherichia coli
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 74 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 75 - DNA, 2676 bp, Escherichia coli
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 76 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 77 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 78 - DNA, 24 bp, artificial sequence
  primer
tcatcactgataacctgattccgg

SEQ ID NO: 79 - DNA, 26 bp, artificial sequence
  primer
cgagtctgttttggcagtcaccttaa

SEQ ID NO: 80 - DNA, 23 bp, artificial sequence
  primer
gagcgtgacgacgtcaacttcct

SEQ ID NO: 81 - DNA, 23 bp, artificial sequence
  primer
cagttcaatgctgaaccacacag

SEQ ID NO: 82 - DNA, 23 bp, artificial sequence
  primer
gaaggttgcgcctacactaagca

SEQ ID NO: 83 - DNA, 23 bp, artificial sequence
  primer
gggagcggcaagattaaaccagt

SEQ ID NO: 84 - DNA, 23 bp, artificial sequence
  primer
tggatcacgtaatcagtacccag

SEQ ID NO: 85 - DNA, 23 bp, artificial sequence
  primer
atccttaactgatcggcattgcc

SEQ ID NO: 86 - DNA, 30 bp, artificial sequence
  primer
ggaattcacacatgaaagctctggtttatc

SEQ ID NO: 87 - DNA, 28 bp, artificial sequence
  primer
gcgtccagggcgtcaaagatcaggcagc

SEQ ID NO: 88 - DNA, 30 bp, artificial sequence
  primer
gacctaggaggtcacacatgaaagctctgg

SEQ ID NO: 89 - DNA, 25 bp, artificial sequence
  primer
cgactctagaggatccccgggtacc

SEQ ID NO: 90 - protein, 602 aa, Escherichia coli
MQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGLAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 91 - DNA, 1809 bp, Escherichia coli
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 92 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW

SEQ ID NO: 93 - DNA, 396 bp, Escherichia coli
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 94 - protein, 119 aa, Escherichia coli
MINPNPKRSDEPVFWGLFGAGGMWSAIIAPVMILLVGILLPLGLFPGDALSYERVLAFAQSFIGRVFLFLMIVLPLWCGLHRMHHAMHDLKIHVPAGKWVFYGLAAILTVVTLIGVVTI

SEQ ID NO: 95 - DNA, 360 bp, Escherichia coli
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa

(19) United States
(12) Patent Application Publication
(10) Pub. No.: US 2011/0136193 A1
(43) Pub. Date: Jun. 9, 2011

(54) METHOD FOR PRODUCING BUTANOL USING EXTRACTIVE FERMENTATION WITH OSMOLYTE ADDITION

(75) Inventors: MICHAEL CHARLES GRADY, Oaklyn, NJ (US); RANJAN PATNAIK, Newark, DE (US)

(73) Assignee: BUTAMAX(TM) ADVANCED BIOFUELS LLC, Wilmington, DE (US)

(21) Appl. No.: 12/952,549

(22) Filed: Nov. 23, 2010

Related U.S. Application Data
(60) Provisional application No. 61/263,522, filed on Nov. 23, 2009.

(51) Int. Cl. C12P 7/16; C07C 29/86

(52) U.S. Cl. 435/160; 568/918

(57) ABSTRACT

A method is provided for producing butanol through microbial fermentation, in which the butanol product is removed during the fermentation by extraction into a water-immiscible organic extractant in the presence of at least one osmolyte at a concentration at least sufficient to increase the butanol partition coefficient relative to that in the presence of the osmolyte concentration of the basal fermentation medium and of an optional fermentable carbon source. The osmolyte may comprise a monosaccharide, a disaccharide, glycerol, sugarcane juice, molasses, polyethylene glycol, dextran, high fructose corn syrup, corn mash, starch, cellulose, and combinations thereof. Also provided is a method and composition for recovering butanol from a fermentation medium.

### CROSS-REFERENCE TO RELATED APPLICATIONS

This application claims the benefit of priority to the U.S. Provisional Patent Application Ser. No. 61/263,522, filed on Nov. 23, 2009, the entirety of which is herein incorporated by reference.

### FIELD OF THE INVENTION

The present invention relates to the field of biofuels. More specifically, the invention relates to a method for producing butanol through microbial fermentation, in which at least one osmolyte is present in the fermentation medium at a concentration at least sufficient to increase the butanol partition coefficient relative to that in the presence of the osmolyte concentration of the basal fermentation medium and of an optional fermentable carbon source, and the butanol product is removed by extraction into a water-immiscible organic extractant.

### BACKGROUND

Butanol is an important industrial chemical with a variety of applications, such as use as a fuel additive, as a blend component to diesel fuel, as a feedstock chemical in the plastics industry, and as a foodgrade extractant in the food and flavor industry. Each year 10 to 12 billion pounds of butanol are produced by petrochemical means. As the need for butanol increases, interest in producing this chemical from renewable resources such as corn, sugar cane, or cellulosic feeds by fermentation is expanding.

In a fermentative process to produce butanol, in situ product removal advantageously reduces butanol inhibition of the microorganism and improves fermentation rates by controlling butanol concentrations in the fermentation broth. Technologies for in situ product removal include stripping, adsorption, pervaporation, membrane solvent extraction, and liquid-liquid extraction. In liquid-liquid extraction, an extractant is contacted with the fermentation broth to partition the butanol between the fermentation broth and the extractant phase. The butanol and the extractant are recovered by a separation process, for example by distillation.

Published Patent Application US 2009/0171129 A1 discloses methods for recovery of C3-C6 alcohols from dilute aqueous solutions, such as fermentation broths. The method includes increasing the activity of the C3-C6 alcohol in a portion of the aqueous solution to at least that of saturation of the C3-C6 alcohol in the portion. According to an embodiment of the invention, increasing the activity of the C3-C6 alcohol may comprise adding a hydrophilic solute to the aqueous solution. Sufficient hydrophilic solute is added to enable the formation of a second liquid phase, either solely by addition of the hydrophilic solute or in combination with other process steps. The added hydrophilic solute may be a salt, an amino acid, a water-soluble solvent, a sugar or combinations of those.

U.S. patent application Ser. No. 12/478,389 filed on Jun. 4, 2009, discloses methods for producing and recovering butanol from a fermentation broth, the methods comprising the step of contacting the fermentation broth with a water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof, to form a two-phase mixture comprising an aqueous phase and a butanol-containing organic phase.

U.S. Provisional Patent Application Nos. 61/168,640; 61/168,642; and 61/168,645; filed concurrently on Apr. 13, 2009; and 61/231,697; 61/231,698; and 61/231,699; filed concurrently on Aug. 6, 2009, disclose methods for producing and recovering butanol from a fermentation medium, the methods comprising the step of contacting the fermentation medium with a water-immiscible organic extractant comprising a first solvent and a second solvent, the first solvent being selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof, and the second solvent being selected from the group consisting of C₇ to C₁₁ alcohols, C₇ to C₁₁ carboxylic acids, esters of C₇ to C₁₁ carboxylic acids, C₇ to C₁₁ aldehydes, and mixtures thereof, to form a two-phase mixture comprising an aqueous phase and a butanol-containing organic phase.

Improved methods for producing and recovering butanol from a fermentation medium are continually sought. A process for in situ product removal of butanol in which osmolyte addition to a fermentation medium provides improved butanol extraction efficiency and acceptable biocompatibility with the microorganism is desired.

### SUMMARY OF THE INVENTION

The present invention provides a method for recovering butanol from a fermentation medium comprising butanol, water, at least one osmolyte, and a genetically modified microorganism that produces butanol from at least one fermentable carbon source. The osmolyte is present in the fermentation medium at a concentration at least sufficient to increase the butanol partition coefficient relative to that in the presence of the osmolyte concentration of the basal fermentation medium and of an optional fermentable carbon source. The present invention also provides methods for the production of butanol using such a microorganism and an added osmolyte. The methods include contacting the fermentation medium with i) a first water-immiscible organic extractant and optionally ii) a second water-immiscible organic extractant, optionally separating the butanol-containing organic phase from the organic phase, and recovering the butanol from the butanol-containing organic phase. In one embodiment of the invention, a method for recovering butanol from a fermentation medium is provided, the method comprising:

a) providing a fermentation medium comprising butanol, water, at least one osmolyte at a concentration at least sufficient to increase the butanol partition coefficient relative to that in the presence of the osmolyte concentration of the basal fermentation medium and of an optional fermentable carbon source, and a genetically modified microorganism that produces butanol from at least one fermentable carbon source;

b) contacting the fermentation medium with i) a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof, and optionally ii) a second water-immiscible organic extractant selected from the group consisting of C₇ to C₂₂ fatty alcohols, C₇ to C₂₂ fatty acids, esters of C₇ to C₂₂ fatty acids, C₇ to C₂₂ fatty aldehydes, C₇ to C₂₂ fatty amides, and mixtures thereof to form a two-phase mixture comprising an aqueous phase and a butanol-containing organic phase;

c) optionally separating the butanol-containing organic phase from the aqueous phase; and

d) optionally, recovering the butanol from the butanol-containing organic phase to produce recovered butanol.

In some embodiments, a portion of the butanol is concurrently removed from the fermentation medium by a process comprising the steps of: a) stripping butanol from the fermentation medium with a gas to form a butanol-containing gas phase; and b) recovering butanol from the butanol-containing gas phase.

According to the methods of the invention, the osmolyte may be added to the fermentation medium, to the first extractant, to the optional second extractant, or to combinations thereof. In some embodiments, the osmolyte comprises a monosaccharide, a disaccharide, glycerol, sugarcane juice, molasses, polyethylene glycol, dextran, high fructose corn syrup, corn mash, starch, cellulose, and combinations thereof. In some embodiments, the osmolyte comprises a monosaccharide selected from the group consisting of sucrose, fructose, glucose, and combinations thereof. In some embodiments, the osmolyte is selected from the group consisting of polyethylene glycol, dextran, corn mash, starch, cellulose, and combinations thereof.

According to the methods of the invention, in some embodiments the genetically modified microorganism is selected from the group consisting of bacteria, cyanobacteria, filamentous fungi, and yeasts. In some embodiments, the bacteria are selected from the group consisting of Zymomonas, Escherichia, Salmonella, Rhodococcus, Pseudomonas, Bacillus, Lactobacillus, Enterococcus, Pediococcus, Alcaligenes, Klebsiella, Paenibacillus, Arthrobacter, Corynebacterium, and Brevibacterium. In some embodiments the yeast is selected from the group consisting of Pichia, Candida, Hansenula, Kluyveromyces, Issatchenkia, and Saccharomyces.

According to the methods of the invention, the first extractant may be selected from the group consisting of oleyl alcohol, behenyl alcohol, cetyl alcohol, lauryl alcohol, myristyl alcohol, stearyl alcohol, oleic acid, lauric acid, myristic acid, stearic acid, methyl myristate, methyl oleate, lauric aldehyde, 1-dodecanol, and a combination of these. In some embodiments, the first extractant comprises oleyl alcohol. In some embodiments, the second extractant may be selected from the group consisting of 1-nonanol, 1-decanol, 1-undecanol, 2-undecanol, 1-nonanal, and a combination of these.

In some embodiments, the butanol is 1-butanol. In some embodiments, the butanol is 2-butanol. In some embodiments, the butanol is isobutanol. In some embodiments, the fermentation medium further comprises ethanol, and the butanol-containing organic phase contains ethanol.

In one embodiment of the invention, a method for the production of butanol is provided, the method comprising:

a) providing a genetically modified microorganism that produces butanol from at least one fermentable carbon source;

b) growing the microorganism in a biphasic fermentation medium comprising an aqueous phase and i) a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof, and optionally ii) a second water-immiscible organic extractant selected from the group consisting of C₇ to C₂₂ alcohols, C₇ to C₂₂-carboxylic acids, esters of C₇ to C₂₂ carboxylic acids, C₇ to C₂₂ aldehydes, C₇ to C₂₂ amides, and mixtures thereof, wherein the biphasic fermentation medium further comprises at least one osmolyte at a concentration at least sufficient to increase the butanol partition coefficient relative to that in the presence of the osmolyte concentration of the basal fermentation medium and of an optional fermentable carbon source, for a time sufficient to allow extraction of the butanol into the organic extractant to form a butanol-containing organic phase;

c) separating the butanol-containing organic phase from the aqueous phase; and

d) optionally, recovering the butanol from the butanol-containing organic phase to produce recovered butanol.

In one embodiment of the invention, a method for the production of butanol is provided, the method comprising:

a) providing a genetically modified microorganism that produces butanol from at least one fermentable carbon source;

b) growing the microorganism in a fermentation medium wherein the microorganism produces the butanol into the fermentation medium to produce a butanol-containing fermentation medium;

c) adding at least one osmolyte to the fermentation medium to provide the osmolyte at a concentration at least sufficient to increase the butanol partition coefficient relative to that in the presence of the osmolyte concentration of the basal fermentation medium and of an optional fermentable carbon source;

d) contacting at least a portion of the butanol-containing fermentation medium with i) a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides and mixtures thereof, and optionally ii) a second water-immiscible organic extractant selected from the group consisting of C₇ to C₂₂ alcohols, C₇ to C₂₂ carboxylic acids, esters of C₇ to C₂₂ carboxylic acids, C₇ to C₂₂ aldehydes, C₇ to C₂₂ amides and mixtures thereof, to form a two-phase mixture comprising an aqueous phase and a butanol-containing organic phase;

e) separating the butanol-containing organic phase from the aqueous phase;

f) optionally, recovering the butanol from the butanol-containing organic phase; and

g) optionally, returning at least a portion of the aqueous phase to the fermentation medium.

In some embodiments, the osmolyte may be added to the fermentation medium in step (c) when the microorganism growth phase slows. In some embodiments, the osmolyte may be added to the fermentation medium in step (c) when the butanol production phase is complete.

In some embodiments, the genetically modified microorganism comprises a modification which inactivates a competing pathway for carbon flow. In some embodiments, the genetically modified microorganism does not produce acetone.

### BRIEF DESCRIPTION OF THE FIGURES AND SEQUENCE DESCRIPTIONS

FIG. 1 schematically illustrates one embodiment of the methods of the invention, in which the first extractant and the second extractant are combined in a vessel prior to contacting with the fermentation medium in a fermentation vessel.

FIG. 2 schematically illustrates one embodiment of the methods of the invention, in which the first extractant and the second extractant are added separately to a fermentation vessel in which the fermentation medium is contacted with the extractants.

FIG. 3 schematically illustrates one embodiment of the methods of the invention, in which the first extractant and the second extractant are added separately to different fermentation vessels.

FIG. 4 schematically illustrates one embodiment of the methods of the invention, in which extraction of the product occurs downstream of the fermentor and the first extractant and the second extractant are combined in a vessel prior to contacting the fermentation medium with the extractants in a different vessel.

FIG. 5 schematically illustrates one embodiment of the methods of the invention, in which extraction of the product occurs downstream of the fermentor and the first extractant and the second extractant are added separately to a vessel in which the fermentation medium is contacted with the extractants.

FIG. 6 schematically illustrates one embodiment of the methods of the invention, in which extraction of the product occurs downstream of the fermentor and the first extractant and the second extractant are added separately to different vessels for contacting with the fermentation medium.

FIG. 7 schematically illustrates one embodiment of the methods of the invention, in which extraction of the product occurs in at least one batch fermentor via co-current flow of a water-immiscible organic extractant at or near the bottom of a fermentation mash to fill the fermentor with extractant which flows out of the fermentor at a point at or near the top of the fermentor.

The following sequences conform with 37 C.F.R. 1.821 1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a bis), and Section 208 and Annex C of the Administrative Instructions).

TABLE 1a
SEQ ID Numbers of Coding Sequences and Proteins

| | SEQ ID NO: | SEQ ID NO: |
|---|---|---|
| Description | Nucleic acid | Amino acid |
| Klebsiella pneumoniae budB (acetolactate | 1 | 2 |
| synthase) | | |
| E. coli ilvC (acetohydroxy acid | 3 | 4 |
| reductoisomerase) | | |
| E. coli ilvD (acetohydroxy acid | 5 | 6 |
| dehydratase) | | |
| Lactococcus lactis kivD (branched-chain α- | 7 (codon | 8 |
| keto acid decarboxylase) | optimized) | |
| Achromobacter xylosoxidans sadB | 9 | 10 |
| (butanol dehydrogenase) | | |
| Bacillus subtilis alsS (acetolactate | 11 | 12 |
| synthase) | | |
| S. cerevisiae ILV5 (acetohydroxy acid | 13 | 14 |
| reductoisomerase; “KARI”) | | |
| Mutant KARI (encoded by Pf5.ilvC-Z4B8) | 15 | 16 |
| Streptococcus mutans ilvD (acetohydroxy | 17 | 18 |
| acid dehydratase) | | |
| Bacillus subtilis kivD (branched-chain keto | 19 (codon | 20 |
| acid decarboxylase) | optimized) | |
| Horse liver alcohol dehydrogenase | 56 (codon | 57 |
| (HADH) | optimized) | |
| E. coli pflB (pyruvate formate lyase) | 71 | 70 |
| E. coli frdB (subunit of fumarate reductase | 73 | 72 |
| enzyme complex) | | |
| E. coli ldhA (lactate dehydrogenase) | 77 | 76 |
| E. coli adhE (alcohol dehydrogenase) | 75 | 74 |
| E. coli frdA (subunit of fumarate reductase | 91 | 90 |
| enzyme complex) | | |
| E. coli frdC (subunit of fumarate reductase | 93 | 92 |
| enzyme complex) | | |
| E. coli frdD (subunit of fumarate reductase | 95 | 94 |
| enzyme complex) | | |

TABLE 1b
SEQ ID Numbers of Sequences used in construction,
Primers and Vectors

| Description | SEQ ID NO: |
|---|---|
| pRS425::GPM-sadB | 63 |
| GPM-sadB-ADHt segment | 21 |
| pUC19-URA3r | 22 |
| 114117-11A | 23 |
| 114117-11B | 24 |
| 114117-11C | 25 |
| 114117-11D | 26 |
| 114117-13A | 27 |
| 114117-13B | 28 |
| 112590-34F | 29 |
| 112590-34G | 30 |
| 112590-34H | 31 |
| 112590-49E | 32 |
| ilvD-FBA1t segment | 33 |
| 114117-27A | 34 |
| 114117-27B | 35 |
| 114117-27C | 36 |
| 114117-27D | 37 |
| 114117-36D | 38 |
| 135 | 39 |
| 112590-30F | 40 |
| URA3r2 template | 41 |
| 114117-45A | 42 |
| 114117-45B | 43 |
| PDC5::KanMXF | 44 |
| PDC5::KanMXR | 45 |
| PDC5kofor | 46 |
| N175 | 47 |
| pLH475-Z4B8 plasmid | 48 |
| CUP1 promoter | 49 |
| CYC1 terminator CYC1-2 | 50 |
| ILV5 promoter | 51 |
| ILV5 terminator | 52 |
| FBA1 promoter | 53 |
| CYC1 terminator | 54 |
| pLH468 plasmid | 55 |
| Vector pNY8 | 58 |
| GPD1 promoter | 59 |
| GPD1 promoter fragment | 60 |
| OT1068 | 61 |
| OT1067 | 62 |
| GPM1 promoter | 64 |
| ADH1 terminator | 65 |
| OT1074 | 66 |
| OT1075 | 67 |
| pRS423 FBA ilvD(Strep) | 68 |
| FBA terminator | 69 |
| pflB CkUp | 78 |
| pflB CkDn | 79 |
| frdB CkUp | 80 |
| frdB CkDn | 81 |
| ldhA CkUp | 82 |
| ldhA CkDn | 83 |
| adhE CkUp | 84 |
| adhE CkDn | 85 |
| N473 | 86 |
| N469 | 87 |
| N695A | 88 |
| N695B | 89 |

### DETAILED DESCRIPTION

The present invention provides methods for recovering butanol from a microbial fermentation medium comprising at least one osmolyte by extraction into a water-immiscible organic extractant to form a two-phase mixture comprising an aqueous phase and a butanol-containing organic phase. The osmolyte is present in the fermentation medium at a concentration at least sufficient to increase the butanol partition coefficient relative to that in the presence of the osmolyte concentration of the basal fermentation medium and of an optional fermentable carbon source. The butanol-containing organic phase is separated from the aqueous phase and the butanol may be recovered. Methods for producing butanol are also provided.

### DEFINITIONS

The following definitions are used in this disclosure.

The term “osmolyte” refers to an organic compound that affects osmosis. An osmolyte is soluble in the solution within a cell, and/or in the surrounding fluid (e.g. fermentation broth), and plays a roll in maintaining cell volume, fluid balance, and water potential.

The term “butanol” refers to 1-butanol, 2-butanol, and/or isobutanol, individually or as mixtures thereof.

The term “water-immiscible” refers to a chemical component, such as an extractant or solvent, which is incapable of mixing with an aqueous solution, such as a fermentation broth, in such a manner as to form one liquid phase.

The term “extractant” as used herein refers to one or more organic solvents which are used to extract butanol from a fermentation broth.

The term “biphasic fermentation medium” refers to a two-phase growth medium comprising a fermentation medium (i.e., an aqueous phase) and a suitable amount of a water-immiscible organic extractant.

The term “organic phase”, as used herein, refers to the non-aqueous phase of a biphasic mixture obtained by contacting a fermentation broth with a water-immiscible organic extractant.

The term “aqueous phase”, as used herein, refers to the phase of a biphasic mixture, obtained by contacting an aqueous fermentation medium with a water-immiscible organic extractant, which comprises water.

The term “In Situ Product Removal” as used herein means the selective removal of a specific fermentation product from a biological process such as fermentation to control the product concentration in the biological process.

The term “fermentation broth” as used herein means the mixture of water, sugars, dissolved solids, suspended solids, microorganisms producing butanol, product butanol and all other constituents of the material held in the fermentation vessel in which product butanol is being made by the reaction of sugars to butanol, water and carbon dioxide (CO₂) by the microorganisms present. The fermentation broth may comprise one or more fermentable carbon sources such as the sugars described herein. The fermentation broth is the aqueous phase in biphasic fermentative extraction. From time to time, as used herein the term “fermentation medium” may be used synonymously with “fermentation broth”.

The term “fermentation vessel” as used herein means the vessel in which the fermentation reaction by which product butanol is made from sugars is carried out. The term “fermentor” may be used synonymously herein with “fermentation vessel”.

The term “fermentable carbon source” refers to a carbon source capable of being metabolized by the microorganisms disclosed herein. Suitable fermentable carbon sources include, but are not limited to, monosaccharides, such as glucose or fructose; disaccharides, such as lactose or sucrose; oligosaccharides; polysaccharides, such as starch or cellulose; one-carbon substrates; and a combination of these, which may be found in the fermentation medium. Sources of fermentable carbon include renewable carbon, that is non-petroleum-based carbon, including carbon from agricultural feedstocks, algae, cellulose, hemicellulose, lignocellulose, or any combination thereof.

The term “fatty acid” as used herein refers to a carboxylic acid having a long, aliphatic chain of C₇ to C₂₂ carbon atoms, which is either saturated or unsaturated.

The term “fatty alcohol” as used herein refers to an alcohol having a long, aliphatic chain of C₇ to C₂₂ carbon atoms, which is either saturated or unsaturated.

The term “fatty aldehyde” as used herein refers to an aldehyde having a long, aliphatic chain of C₇ to C₂₂ carbon atoms, which is either saturated or unsaturated.

The term “fatty amide” as used herein refers to an amide having a long, aliphatic chain of C₁₂ to C₂₂ carbon atoms, which is either saturated or unsaturated.

The term “partition coefficient”, abbreviated herein as Kₚ, means the ratio of the concentration of a compound in the two phases of a mixture of two immiscible solvents at equilibrium. A partition coefficient is a measure of the differential solubility of a compound between two immiscible solvents. As used herein, the term “partition coefficient for butanol” refers to the ratio of concentrations of butanol between the organic phase comprising the extractant and the aqueous phase comprising the fermentation medium. Partition coefficient, as used herein, is synonymous with the term distribution coefficient.

The term “separation” as used herein is synonymous with “recovery” and refers to removing a chemical compound from an initial mixture to obtain the compound in greater purity or at a higher concentration than the purity or concentration of the compound in the initial mixture.

The term “butanol biosynthetic pathway” as used herein refers to an enzyme pathway to produce 1-butanol, 2-butanol, or isobutanol.

The term “1-butanol biosynthetic pathway” as used herein refers to an enzyme pathway to produce 1-butanol from acetyl-coenzyme A (acetyl-CoA).

The term “2-butanol biosynthetic pathway” as used herein refers to an enzyme pathway to produce 2-butanol from pyruvate.

The term “isobutanol biosynthetic pathway” as used herein refers to an enzyme pathway to produce isobutanol from pyruvate.

The term “effective titer” as used herein, refers to the total amount of butanol produced by fermentation per liter of fermentation medium. The total amount of butanol includes: (i) the amount of butanol in the fermentation medium; (ii) the amount of butanol recovered from the organic extractant; and (iii) the amount of butanol recovered from the gas phase, if gas stripping is used.

The term “effective rate” as used herein, refers to the total amount of butanol produced by fermentation per liter of fermentation medium per hour of fermentation.

The term “effective yield” as used herein, refers to the amount of butanol produced per unit of fermentable carbon substrate consumed by the biocatalyst during fermentation.

The term “aerobic conditions” as used herein means growth conditions in the presence of oxygen.

The term “microaerobic conditions” as used herein means growth conditions with low levels of oxygen (i.e., below normal atmospheric oxygen levels).

The term “anaerobic conditions” as used herein means growth conditions in the absence of oxygen.

The term “minimal media” as used herein refers to growth media that contain the minimum nutrients possible for growth, generally without the presence of amino acids. A minimal medium typically contains a fermentable carbon source and various salts, which may vary among microorganisms and growing conditions; these salts generally provide essential elements such as magnesium, nitrogen, phosphorus, and sulfur to allow the microorganism to synthesize proteins and nucleic acids.

The term “defined media” as used herein refers to growth media that have known quantities of all ingredients present, e.g., a defined carbon source and nitrogen source, and trace elements and vitamins required by the microorganism.

The term “biocompatibility” as used herein refers to the measure of the ability of a microorganism to utilize glucose in the presence of an extractant. A biocompatible extractant permits the microorganism to utilize glucose. A non-biocompatible (that is, a biotoxic) extractant does not permit the microorganism to utilize glucose, for example at a rate greater than about 25% of the rate when the extractant is not present.

The term, “° C.” means degrees Celsius.

The term “OD” means optical density.

The term “OD₆₀₀” refers to the optical density at a wavelength of 600 nm.

The term ATCC refers to the American Type Culture Collection, Manassas, Va.

The term “sec” means second(s).

The term “min” means minute(s).

The term “h” means hour(s).

The term “mL” means milliliter(s).

The term “L” means liter.

The term “g” means grams.

The term “mmol” means millimole(s).

The term “M” means molar.

The term “μL” means microliter.

The term “μg” means microgram.

The term “μg/mL” means microgram per liter.

The term “mL/min” means milliliters per minute.

The term “g/L” means grams per liter.

The term “g/L/h” means grams per liter per hour.

The term “mmol/min/mg” means millimole per minute per milligram.

The term “temp” means temperature.

The term “rpm” means revolutions per minute.

The term “HPLC” means high pressure gas chromatography.

The term “GC” means gas chromatography.

All publications, patents, patent applications, and other references mentioned herein are expressly incorporated by reference in their entireties for all purposes. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### Genetically Modified Microorganisms

Microbial hosts for butanol production may be selected from bacteria, cyanobacteria, filamentous fungi and yeasts. The microbial host used should be tolerant to the butanol product produced, so that the yield is not limited by toxicity of the product to the host. The selection of a microbial host for butanol production is described in detail below.

Microbes that are metabolically active at high titer levels of butanol are not well known in the art. Although butanol-tolerant mutants have been isolated from solventogenic Clostridia, little information is available concerning the butanol tolerance of other potentially useful bacterial strains. Most of the studies on the comparison of alcohol tolerance in bacteria suggest that butanol is more toxic than ethanol (de Cavalho et al., Microsc. Res. Tech. 64:215-22 (2004) and Kabelitz et al., FEMS Microbiol. Lett. 220:223-227 (2003)). Tomas et al. (J. Bacteriol. 186:2006-2018 (2004)) report that the yield of 1-butanol during fermentation in Clostridium acetobutylicum may be limited by butanol toxicity. The primary effect of 1-butanol on Clostridium acetobutylicum is disruption of membrane functions (Hermann et al., Appl. Environ. Microbiol. 50:1238-1243 (1985)).

The microbial hosts selected for the production of butanol should be tolerant to butanol and should be able to convert carbohydrates to butanol using the introduced biosynthetic pathway as described below. The criteria for selection of suitable microbial hosts include the following: intrinsic tolerance to butanol, high rate of carbohydrate utilization, availability of genetic tools for gene manipulation, and the ability to generate stable chromosomal alterations.

Suitable host strains with a tolerance for butanol may be identified by screening based on the intrinsic tolerance of the strain. The intrinsic tolerance of microbes to butanol may be measured by determining the concentration of butanol that is responsible for 50% inhibition of the growth rate (IC50) when grown in a minimal medium. The IC50 values may be determined using methods known in the art. For example, the microbes of interest may be grown in the presence of various amounts of butanol and the growth rate monitored by measuring the optical density at 600 nanometers. The doubling time may be calculated from the logarithmic part of the growth curve and used as a measure of the growth rate. The concentration of butanol that produces 50% inhibition of growth may be determined from a graph of the percent inhibition of growth versus the butanol concentration. Preferably, the host strain should have an IC50 for butanol of greater than about 0.5%. More suitable is a host strain with an IC50 for butanol that is greater than about 1.5%. Particularly suitable is a host strain with an IC50 for butanol that is greater than about 2.5%.

The microbial host for butanol production should also utilize glucose and/or other carbohydrates at a high rate. Most microbes are capable of utilizing carbohydrates. However, certain environmental microbes cannot efficiently use carbohydrates, and therefore would not be suitable hosts.

The ability to genetically modify the host is essential for the production of any recombinant microorganism. Modes of gene transfer technology that may be used include by electroporation, conjugation, transduction or natural transformation. A broad range of host conjugative plasmids and drug resistance markers are available. The cloning vectors used with an organism are tailored to the host organism based on the nature of antibiotic resistance markers that can function in that host.

The microbial host also may be manipulated in order to inactivate competing pathways for carbon flow by inactivating various genes. This requires the availability of either transposons or chromosomal integration vectors to direct inactivation. Additionally, production hosts that are amenable to chemical mutagenesis may undergo improvements in intrinsic butanol tolerance through chemical mutagenesis and mutant screening.

As an example of inactivation of competing pathways for carbon flow, pyruvate decarboxylase may be reduced or eliminated (see, for example, US Published Patent Application No. 20090305363.) In embodiments, butanol is the major product of the microorganism. In embodiments, the microorganism does not produce acetone.

Based on the criteria described above, suitable microbial hosts for the production of butanol include, but are not limited to, members of the genera, Zymomonas, Escherichia, Salmonella, Rhodococcus, Pseudomonas, Bacillus, Lactobacillus, Enterococcus, Pediococcus, Alcaligenes, Klebsiella, Paenibacillus, Arthrobacter, Corynebacterium, Brevibacterium, Pichia, Candida, Hansenula, Kluyveromyces, Issatchenkia and Saccharomyces. Preferred hosts include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Pediococcus pentosaceus, Pediococcus acidilactici, Bacillus subtilis and Saccharomyces cerevisiae.

Microorganisms mentioned above may be genetically modified to convert fermentable carbon sources into butanol, specifically 1-butanol, 2-butanol, or isobutanol, using methods known in the art. Suitable microorganisms include Escherichia, Lactobacillus, and Saccharomyces. Suitable microorganisms include E. coli, L. plantarum and S. cerevisiae. Additionally, the microorganism may be a butanol-tolerant strain of one of the microorganisms listed above that is isolated using the method described by Bramucci et al. (U.S. patent application Ser. No. 11/761,497; and WO 2007/146377). An example of one such strain is Lactobacillus plantarum strain PN0512 (ATCC: PTA-7727, biological deposit made Jul. 12, 2006 for U.S. patent application Ser. No. 11/761,497).

Suitable biosynthetic pathways for production of butanol are known in the art, and certain suitable pathways are described herein. In some embodiments, the butanol biosynthetic pathway comprises at least one gene that is heterologous to the host cell. In some embodiments, the butanol biosynthetic pathway comprises more than one gene that is heterologous to the host cell. In some embodiments, the butanol biosynthetic pathway comprises heterologous genes encoding polypeptides corresponding to every step of a biosynthetic pathway.

Likewise, certain suitable proteins having the ability to catalyze indicated substrate to product conversions are described herein and other suitable proteins are provided in the art. For example, US Patent Application Publication Nos. US20080261230, US20090163376, and US20100197519 describe acetohydroxy acid isomeroreductases as does U.S. application Ser. No. 12/893,077, filed on Sep. 29, 2010; US Patent Application Publication No. 20100081154 describes dihydroxyacid dehydratases; alcohol dehydrogenases are described in US Patent Application Publication No. US20090269823 and U.S. Provisional Patent Application No. 61/290,636.

Microorganisms can be genetically modified to contain a 1-butanol biosynthetic pathway to produce 1-butanol. Suitable modifications include those described by Donaldson et al. in WO 2007/041269, incorporated herein by reference. For example, the microorganism may be genetically modified to express a 1-butanol biosynthetic pathway comprising the following enzyme-catalyzed substrate to product conversions:

a) acetyl-CoA to acetoacetyl-CoA;

b) acetoacetyl-CoA to 3-hydroxybutyryl-CoA;

c) 3-hydroxybutyryl-CoA to crotonyl-CoA;

d) crotonyl-CoA to butyryl-CoA;

e) butyryl-CoA to butyraldehyde; and

f) butyraldehyde to a-butanol.

The microorganisms may also be genetically modified to express a 2-butanol biosynthetic pathway to produce 2-butanol. Suitable modifications include those described by Donaldson et al. in U.S. Patent Application Publication Nos. 2007/0259410 and 2007/0292927, and PCT Application Publication Nos. WO 2007/130518 and WO 2007/130521. For example, in one embodiment the microorganism may be genetically modified to express a 2-butanol biosynthetic pathway comprising the following enzyme-catalyzed substrate to product conversions:

a) pyruvate to alpha-acetolactate;

b) alpha-acetolactate to acetoin;

c) acetoin to 2,3-butanediol;

d) 2,3-butanediol to 2-butanone; and

e) 2-butanone to 2-butanol.

The microorganisms may also be genetically modified to express an isobutanol biosynthetic pathway to produce isobutanol. Suitable modifications include those described by Donaldson et al. in U.S. Patent Application Publication Nos. 2007/0092957 and WO 2007/050671. For example, the microorganism may be genetically modified to contain an isobutanol biosynthetic pathway comprising the following enzyme-catalyzed substrate to product conversions:

a) pyruvate to acetolactate;

b) acetolactate to 2,3-dihydroxyisovalerate;

c) 2,3-dihydroxyisovalerate to α-ketoisovalerate;

d) α-ketoisovalerate to isobutyraldehyde; and

e) isobutyraldehyde to isobutanol.

The Escherichia coli strain may comprise: (a) an isobutanol biosynthetic pathway encoded by the following genes: budB (SEQ ID NO: 1) from Klebsiella pneumoniae encoding acetolactate synthase (given as SEQ ID NO: 2), ilvC (given as SEQ ID NO: 3) from E. coli encoding acetohydroxy acid reductoisomerase (given as SEQ ID NO: 4), ilvD (given as SEQ ID NO: 5) from E. coli encoding acetohydroxy acid dehydratase (given as SEQ iD NO: 6), kivD (given as SEQ ID NO: 7) from Lactococcus lactis encoding the branched-chain keto acid decarboxylase (given as SEQ ID NO: 8), and sadB (given as SEQ ID NO: 9) from Achromobacter xylosoxidans encoding a butanol dehydrogenase (given as SEQ ID NO: 10). The enzymes encoded by the genes of the isobutanol biosynthetic pathway catalyze the substrate to product conversions for converting pyruvate to isobutanol, as described above. Specifically, acetolactate synthase catalyzes the conversion of pyruvate to acetolactate, acetohydroxy acid reductoisomerase catalyzes the conversion of acetolactate to 2,3-dihydroxyisovalerate, acetohydroxy acid dehydratase catalyzes the conversion of 2,3-dihydroxyisovalerate to α-ketoisovalerate, branched-chain keto acid decarboxylase catalyzes the conversion of α-ketoisovalerate to isobutyraldehyde, and butanol dehydrogenase catalyzes the conversion of isobutyraldehyde to isobutanol. This recombinant Escherichia coli strain can be constructed using methods known in the art (see copending U.S. patent application Ser. Nos. 12/478,389 and 12/477,946) and/or described herein below. It is contemplated that suitable strains may be constructed comprising a sequence having at least about 70-75% identity, at least about 75-80%, at least about 80-85% identity, or at least about 85-90% identity to protein sequences described herein.

The Escherichia coli strain may comprise deletions of the following genes to eliminate competing pathways that limit isobutanol production, pflB, given as SEQ ID No: 71, (encoding for pyruvate formate lyase) ldhA, given as SEQ IS NO: 73, (encoding for lactate dehydrogenase), adhE, given as SEQ IS NO: 77, (encoding for alcohol dehydrogenase), and at least one gene comprising the frdABCD operon (encoding for fumarate reductase), specifically, frdA, given as SEQ ID NO: 90, frdB, given as SEQ ID NO: 75, frdC, given as SEQ ID NO: 92, and frdD, given as SEQ ID NO: 94.

The Saccharomyces cerevisiae strain may comprise: an isobutanol biosynthetic pathway encoded by the following genes: alsS coding region from Bacillus subtilis (SEQ ID NO: 11) encoding acetolactate synthase (SEQ ID NO: 12), ILV5 from S. cerevisiae (SEQ ID NO: 13) encoding acetohydroxy acid reductoisomerase (KARI; SEQ ID NO: 14) and/or a mutant KARI such as encoded by Pf5.IlvC-Z4B8 (SEQ ID NO: 15; protein SEQ ID NO: 16), ilvD from Streptococcus mutans (SEQ ID NO: 17) encoding acetohydroxy acid dehydratase (SEQ ID NO: 18), kivD from Bacillus subtilis (codon optimized sequence given as SEQ ID NO: 19) encoding the branched-chain keto acid decarboxylase (SEQ ID NO: 20), and sadB from Achromobacter xylosoxidans (SEQ ID NO: 9) encoding a butanol dehydrogenase (SEQ ID NO: 10). The enzymes encoded by the genes of the isobutanol biosynthetic pathway catalyze the substrate to product conversions for converting pyruvate to isobutanol, as described herein. It is contemplated that suitable strains may be constructed comprising a sequence having at least about 70-75% identity, at least about 75-80%, at least about 80-85% identity, or at least about 85-90% identity to amino acid sequences described herein.

A yeast strain expressing an isobutanol pathway with acetolactate synthase (ALS) activity in the cytosol and has deletions of the endogenous pyruvate decarboxylase (PDC) genes is described in U.S. patent application Ser. No. 12/477,942. This combination of cytosolic ALS and reduced PDC expression has been found to greatly increase flux from pyruvate to acetolactate, which then flows to the pathway for production of isobutanol. Such a recombinant Saccharomyces cerevisiae strain can be constructed using methods known in the art and/or described herein. Other suitable yeast strains are known in the art. Additional examples are provided in U.S. Provisional Application Ser. Nos. 61/379,546, 61/380,563, and U.S. application Ser. No. 12/893,089.

Additional modifications suitable for microorganisms used in conjunction with the processes provided herein include modifications to reduce glycerol-3-phosphate dehydrogenase activity as described in US Patent Application Publication No. 20090305363, modifications to a host cell that provide for increased carbon flux through an Entner-Doudoroff Pathway or reducing equivalents balance as described in US Patent Application Publication No. 20100120105. Yeast strains with increased activity of heterologous proteins that require binding of an Fe—S cluster for their activity are described in US Patent Application Publication No. 20100081179. Other modifications include modifications in an endogenous polynucleotide encoding a polypeptide having dual-role hexokinase activity, described in U.S. Provisional Application No. 61/290,639, integration of at least one polynucleotide encoding a polypeptide that catalyzes a step in a pyruvate-utilizing biosynthetic pathway described in U.S. Provisional Application No. 61/380,563.

Additionally, host cells comprising at least one deletion, mutation, and/or substitution in an endogenous gene encoding a polypeptide affecting Fe—S cluster biosynthesis are described in U.S. Provisional Patent Application No. 61/305,333, and host cells comprising a heterologous polynucleotide encoding a polypeptide with phosphoketolase activity and host cells comprising a heterologous polynucleotide encoding a polypeptide with phosphotransacetylase activity are described in U.S. Provisional Patent Application No. 61/356,379.

### Construction of a Suitable Yeast Strain

NGI-049 is an example of a suitable Saccharomyces cerevisiae strain. NGI-049 is a strain with insertion-inactivation of endogenous PDC1, PDC5, and PDC6 genes, and containing expression vectors pLH475-Z4B8 and pLH468. PDC1, PDC5, and PDC6 genes encode the three major isozymes of pyruvate decarboxylase. The strain expresses genes encoding enzymes for an isobutanol biosynthetic pathway that are integrated or on plasmids. Construction of the NGI-049 strain is provided herein.

Endogenous pyruvate decarboxylase activity in yeast converts pyruvate to acetaldehyde, which is then converted to ethanol or to acetyl-CoA via acetate. Therefore, endogenous pyruvate decarboxylase activity is a target for reduction or elimination of byproduct formation.

Examples of other yeast strains with reduced pyruvate decarboxylase activity due to disruption of pyruvate decarboxylase encoding genes have been reported such as for Saccharomyces in Flikweert et al. (Yeast (1996) 12:247-257), for Kluyveromyces in Bianchi et al. (Mol. Microbiol. (1996) 19(1):27-36), and disruption of the regulatory gene in Hohmann, (Mol Gen Genet. (1993) 241:657-666). Saccharomyces strains having no pyruvate decarboxylase activity are available from the ATCC (Accession #200027 and #200028).

Construction of pdc6::GPMp1-sadB Integration Cassette and PDC6 Deletion:

A pdc6::GPM1p-sadB-ADH1t-URA3r integration cassette was made by joining the GPM-sadB-ADHt segment (SEQ ID NO: 21) from pRS425::GPM-sadB (SEQ ID NO: 63) to the URA3r gene from pUC19-URA3r. pUC19-URA3r (SEQ ID NO: 22) contains the URA3 marker from pRS426 (ATCC # 77107) flanked by 75 bp homologous repeat sequences to allow homologous recombination in vivo and removal of the URA3 marker. The two DNA segments were joined by SOE PCR (as described by Horton et al. (1989) Gene 77:61-68) using as template pRS425::GPM-sadB and pUC19-URA3r plasmid DNAs, with Phusion DNA polymerase (New England Biolabs Inc., Beverly, Mass.; catalog no. F-540S) and primers 114117-11A through 114117-11D (SEQ ID NOs: 23, 24, 25 and 26), and 114117-13A and 114117-13B (SEQ ID NOs: 27 and 28).

The outer primers for the SOE PCR (114117-13A and 114117-13B) contained 5′ and 3′˜50 bp regions homologous to regions upstream and downstream of the PDC6 promoter and terminator, respectively. The completed cassette PCR fragment was transformed into BY4700 (ATCC # 200866) and transformants were maintained on synthetic complete media lacking uracil and supplemented with 2% glucose at 30° C. using standard genetic techniques (Methods in Yeast Genetics, 2005, Cold Spring Harbor Laboratory Press, Cold Spring Harbor, N.Y., pp. 201-202). Transformants were screened by PCR using primers 112590-34G and 112590-34H (SEQ ID NOs: 30 and 31), and 112590-34F and 112590-49E (SEQ ID NOs: 29 and 32) to verify integration at the PDC6 locus with deletion of the PDC6 coding region. The URA3r marker was recycled by plating on synthetic complete media supplemented with 2% glucose and 5-FOA at 30° C. following standard protocols. Marker removal was confirmed by patching colonies from the 5-FOA plates onto SD-URA media to verify the absence of growth. The resulting identified strain has the genotype: BY4700 pdc6::P_{GPM1}-sadB-ADH1t.

Construction of pdc1::PDC1-ilvD Integration Cassette and PDC1 Deletion:

A pdc1::PDC1p-ilvD-FBA1t-URA3r integration cassette was made by joining the ilvD-FBA1t segment (SEQ ID NO: 33) from pLH468 to the URA3r gene from pUC19-URA3r by SOE PCR (as described by Horton et al. (1989) Gene 77:61-68) using as template pLH468 and pUC19-URA3r plasmid DNAs, with Phusion DNA polymerase (New England Biolabs Inc., Beverly, Mass.; catalog no. F-5405) and primers 114117-27A through 114117-27D (SEQ ID NOs: 34, 35, 36 and 37).

The outer primers for the SOE PCR (114117-27A and 114117-27D) contained 5′ and 3′˜50 bp regions homologous to regions downstream of the PDC1 promoter and downstream of the PDC1 coding sequence. The completed cassette PCR fragment was transformed into BY4700 pdc6::P_{GPM1}-sadB-ADH1t and transformants were maintained on synthetic complete media lacking uracil and supplemented with 2% glucose at 30° C. using standard genetic techniques (Methods in Yeast Genetics, 2005, Cold Spring Harbor Laboratory Press, Cold Spring Harbor, N.Y., pp. 201-202). Transformants were screened by PCR using primers 114117-36D and 135 (SEQ ID NOs 38 and 39), and primers 112590-49E and 112590-30F (SEQ ID NOs 32 and 40) to verify integration at the PDC1 locus with deletion of the PDC1 coding sequence. The URA3r marker was recycled by plating on synthetic complete media supplemented with 2% glucose and 5-FOA at 30° C. following standard protocols. Marker removal was confirmed by patching colonies from the 5-FOA plates onto SD-URA media to verify the absence of growth. The resulting identified strain “NYLA67” has the genotype: BY4700 pdc6::GPM1p-sadB-ADH1t pdc1::PDC1p-ilvD-FBA1t.

### HIS3 Deletion

To delete the endogenous HIS3 coding region, a his3::URA3r2 cassette was PCR-amplified from URA3r2 template DNA (SEQ ID NO; 41). URA3r2 contains the URA3 marker from pRS426 (ATCC # 77107) flanked by 500 bp homologous repeat sequences to allow homologous recombination in vivo and removal of the URA3 marker. PCR was done using Phusion DNA polymerase and primers 114117-45A and 114117-45B (SEQ ID NOs: 42 and 43) which generated a ˜2.3 kb PCR product. The HIS3 portion of each primer was derived from the 5′ region upstream of the HIS3 promoter and 3′ region downstream of the coding region such that integration of the URA3r2 marker results in replacement of the HIS3 coding region. The PCR product was transformed into NYLA67 using standard genetic techniques (Methods in Yeast Genetics, 2005, Cold Spring Harbor Laboratory Press, Cold Spring Harbor, N.Y., pp. 201-202) and transformants were selected on synthetic complete media lacking uracil and supplemented with 2% glucose at 30° C. Transformants were screened to verify correct integration by replica plating of transformants onto synthetic complete media lacking histidine and supplemented with 2% glucose at 30° C. The URA3r marker was recycled by plating on synthetic complete media supplemented with 2% glucose and 5-FOA at 30° C. following standard protocols. Marker removal was confirmed by patching colonies from the 5-FOA plates onto SD-URA media to verify the absence of growth. The resulting identified strain “NYLA73” has the genotype: BY4700 pdc6::GPM1p-sadB-ADH1t pdc1::PDC1p-ilvD-FBA1t Δhis3.

Construction of pdc5::kanMX Integration Cassette and PDC5 Deletion:

A pdc5::kanMX4 cassette was PCR-amplified from strain YLR134W chromosomal DNA (ATCC No. 4034091) using Phusion DNA polymerase and primers PDC5::KanMXF and PDC5::KanMXR (SEQ ID NOs: 44 and 45) which generated a ˜2.2 kb PCR product. The PDC5 portion of each primer was derived from the 5′ region upstream of the PDC5 promoter and 3′ region downstream of the coding region such that integration of the kanMX4 marker results in replacement of the PDC5 coding region. The PCR product was transformed into NYLA73 using standard genetic techniques (Methods in Yeast Genetics, 2005, Cold Spring Harbor Laboratory Press, Cold Spring Harbor, N.Y., pp. 201-202) and transformants were selected on YP media supplemented with 1% ethanol and geneticin (200 μg/ml) at 30° C. Transformants were screened by PCR to verify correct integration at the PDC locus with replacement of the PDC5 coding region using primers PDC5kofor and N175 (SEQ ID NOs: 46 and 47). The identified correct transformants have the genotype: BY4700 pdc6::GPM1p-sadB-ADH1t pdc1::PDC1p-ilvD-FBA1t Δhis3 pdc5::kanMX4.

pLH475-Z4B8 Construction

The pLH475-Z4B8 plasmid (SEQ ID NO: 48) was constructed for expression of ALS and KARI in yeast. pLH475-Z4B8 is a pHR81 vector (ATCC #87541) containing the following chimeric genes:

1) the CUP1 promoter (SEQ ID NO: 49), acetolactate synthase coding region from Bacillus subtilis (AlsS; SEQ ID NO: 11; protein SEQ ID NO: 12) and CYC1 terminator (CYC1-2; SEQ ID NO: 50);
2) an ILV5 promoter (SEQ ID NO: 51), Pf5.IlvC-Z4B8 coding region (SEQ ID NO: 15; protein SEQ ID NO: 16) and ILV5 terminator (SEQ ID NO: 52);
and 3) the FBA1 promoter (SEQ ID NO: 53), S. cerevisiae KARI coding region (ILV5; SEQ ID NO: 13; protein SEQ ID NO: 14) and CYC1 terminator (SEQ ID NO: 54).

The Pf5.IlvC-Z4B8 coding region is a sequence encoding KARI derived from Pseudomonas fluorescens but containing mutations, that was described in US Patent Application Publication No. US20090163376, which is herein incorporated by reference. The Pf5.IlvC-Z4B8 encoded KARI (SEQ ID NO: 16) has the following amino acid changes as compared to the natural Pseudomonas fluorescens KARI:

C33L: cysteine at position 33 changed to leucine,
R47Y: arginine at position 47 changed to tyrosine,
S50A: serine at position 50 changed to alanine,
T52D: threonine at position 52 changed to asparagine,
V53A: valine at position 53 changed to alanine,
L61F: leucine at position 61 changed to phenylalanine,
T80I: threonine at position 80 changed to isoleucine,
A156V: alanine at position 156 changed to threonine, and
G170A: glycine at position 170 changed to alanine.

The Pf5.IlvC-Z4B8 coding region was synthesized by DNA 2.0 (Palo Alto, Calif.; SEQ ID NO: 15) based on codons that were optimized for expression in Saccharomyces cerevisiae.

Expression Vector pLH468

The pLH468 plasmid (SEQ ID NO: 55) was constructed for expression of DHAD, KivD and HADH in yeast.

Coding regions for B. subtilis ketoisovalerate decarboxylase (KivD) and Horse liver alcohol dehydrogenase (HADH) were synthesized by DNA2.0 based on codons that were optimized for expression in Saccharomyces cerevisiae (SEQ ID NO: 19 and 56, respectively) and provided in plasmids pKivDy-DNA2.0 and pHadhy-DNA2.0. The encoded proteins are SEQ ID NOs 20 and 57, respectively. Individual expression vectors for KivD and HADH were constructed. To assemble pLH467 (PRS426::P_{GPD1}-kivDy-GPD1t), vector pNY8 (SEQ ID NO: 58; also named pRS426.GPD-ald-GPDt, described in US Patent App. Pub. US20080182308, Example 17, which is herein incorporated by reference) was digested with AscI and SfiI enzymes, thus excising the GPD1 promoter (SEQ ID NO: 59) and the ald coding region. A GPD1 promoter fragment (GPD1-2; SEQ ID NO: 60) from pNY8 was PCR amplified to add an AscI site at the 5′ end, and an SpeI site at the 3′ end, using 5′ primer OT1068 and 3′ primer OT1067 (SEQ ID NOs: 61 and 62). The AscI/SfiI digested pNY8 vector fragment was ligated with the GPD1 promoter PCR product digested with AscI and SpeI, and the SpeI-SfiI fragment containing the codon optimized kivD coding region isolated from the vector pKivD-DNA2.0. The triple ligation generated vector pLH467 (pRS426::P_{GPD1}-kivDy-GPD1t). pLH467 was verified by restriction mapping and sequencing.

pLH435 (pRS425::P_{GPM1}-Hadhy-ADH1t) was derived from vector pRS425::GPM-sadB (SEQ ID NO: 63) which is described in U.S. patent application Ser. No. 12/477,942, Example 3, which is herein incorporated by reference. pRS425::GPM-sadB is the pRS425 vector (ATCC #77106) with a chimeric gene containing the GPM1 promoter (SEQ ID NO: 64), coding region from a butanol dehydrogenase of Achromobacter xylosoxidans (sadB; SEQ ID NO: 9; protein SEQ ID NO: 10: disclosed in US Patent App. Publication No. US20090269823), and ADH1 terminator (SEQ ID NO: 65). pRS425::GPMp-sadB contains BbvI and PacI sites at the 5′ and 3′ ends of the sadB coding region, respectively. A NheI site was added at the 5′ end of the sadB coding region by site-directed mutagenesis using primers OT1074 and OT1075 (SEQ ID NO: 66 and 67) to generate vector pRS425-GPMp-sadB-NheI, which was verified by sequencing. pRS425::P_{GPM1}-sadB-NheI was digested with NheI and PacI to drop out the sadB coding region, and ligated with the NheI-PacI fragment containing the codon optimized HADH coding region from vector pHadhy-DNA2.0 to create pLH435.

To combine KivD and HADH expression cassettes in a single vector, yeast vector pRS411 (ATCC # 87474) was digested with SacI and NotI, and ligated with the SacI-SalI fragment from pLH467 that contains the P_{GPD1}-kivDy-GPD1t cassette together with the SalI-NotI fragment from pLH435 that contains the P_{GPM1}-Hadhy-ADH1t cassette in a triple ligation reaction. This yielded the vector pRS411::P_{GPD1}-kivDy-P_{GPM1}-Hadhy (pLH441), which was verified by restriction mapping.

In order to generate a co-expression vector for all three genes in the lower isobutanol pathway: ilvD, kivDy and Hadhy, we used pRS423 FBA ilvD (Strep) (SEQ ID NO: 68), which is described in U.S. patent application Ser. No. 12/569,636 as the source of the IlvD gene. This shuttle vector contains an F1 origin of replication (nt 1423 to 1879) for maintenance in E. coli and a 2 micron origin (nt 8082 to 9426) for replication in yeast. The vector has an FBA promoter (nt 2111 to 3108; SEQ ID NO: 53;) and FBA terminator (nt 4861 to 5860; SEQ ID NO: 69). In addition, it carries the His marker (nt 504 to 1163) for selection in yeast and ampicillin resistance marker (nt 7092 to 7949) for selection in E. coli. The ilvD coding region (nt 3116 to 4828; SEQ ID NO: 17; protein SEQ ID NO: 18) from Streptococcus mutans UA159 (ATCC #700610) is between the FBA promoter and FBA terminator forming a chimeric gene for expression. In addition there is a lumio tag fused to the ilvD coding region (nt 4829-4849).

The first step was to linearize pRS423 FBA ilvD (Strep) (also called pRS423-FBA(SpeI)-IlvD(Streptococcus mutans)-Lumio) with SacI and SacII (with SacII site blunt ended using T4 DNA polymerase), to give a vector with total length of 9,482 bp. The second step was to isolate the kivDy-hADHy cassette from pLH441 with SacI and KpnI (with KpnI site blunt ended using T4 DNA polymerase), which gives a 6,063 bp fragment. This fragment was ligated with the 9,482 bp vector fragment from pRS423-FBA(SpeI)-IlvD(Streptococcus mutans)-Lumio. This generated vector pLH468 (pRS423::P_{FBA1}-ilvD(Strep)Lumio-FBA1t-P_{GPD1}-kivDy-GPD1t-P_{GPM1}-hadhy-ADH1t), which was confirmed by restriction mapping and sequencing.

Plasmid vectors pLH468 and pLH475-Z4B8 were simultaneously transformed into strain BY4700 pdc6::GPM1p-sadB-ADH1t pdc1::PDC1p-ilvD-FBA1t Δhis3 pdc5::kanMX4 using standard genetic techniques (Methods in Yeast Genetics, 2005, Cold Spring Harbor Laboratory Press, Cold Spring Harbor, N.Y.) and the resulting strain was maintained on synthetic complete media lacking histidine and uracil, and supplemented with 1% ethanol at 30° C. The resulting strain was named NGI-049.

Construction of a Suitable E. coli Strain

NGCI-031 is an example of a suitable E. coli strain. NGCI-031 is a strain containing an isobutanol biosynthetic pathway and deletions of pflB, frdB, ldhA, and adhE genes. Construction of the NGCI-031 strain is provided herein.

Construction of an E. coli Strain Having Deletions of pflB, frdB, ldhA, and adhE Genes

Provided herein is a suitable method for deleting pflB, frdB, ldhA, and adhE genes from E. coli. The Keio collection of E. coli strains (Baba et al., Mol. Syst. Biol., 2:1-11, 2006) was used for production of eight of the knockouts. The Keio collection (available from NBRP at the National Institute of Genetics, Japan) is a library of single gene knockouts created in strain E. coli BW25113 by the method of Datsenko and Wanner (Datsenko, K. A. & Wanner, B. L., Proc Natl Acad. Sci., USA, 97: 6640-6645, 2000). In the collection, each deleted gene was replaced with a FRT-flanked kanamycin marker that was removable by Flp recombinase. The E. coli strain carrying multiple knockouts was constructed by moving the knockout-kanamycin marker from the Keio donor strain by bacteriophage P1 transduction to a recipient strain. After each P1 transduction to produce a knockout, the kanamycin marker was removed by Flp recombinase. This markerless strain acted as the new recipient strain for the next P1 transduction. One of the described knockouts was constructed directly in the strain using the method of Datsenko and Wanner (supra) rather than by P1 transduction.

The 4KO E. coli strain was constructed in the Keio strain JW0886 by P1ᵥᵢᵣ transductions with P1 phage lysates prepared from three Keio strains. The Keio strains used are listed below:
JW0886: the kan marker is inserted in the pflB
JW4114: the kan marker is inserted in the frdB
JW1375: the kan marker is inserted in the ldhA
JW1228: the kan marker is inserted in the adhE

[Sequences corresponding to the inactivated genes are: pflB (SEQ ID NO: 71), frdB (SEQ ID NO: 73), ldhA (SEQ ID NO: 77), adhE (SEQ ID NO: 75).]

Removal of the FRT-flanked kanamycin marker from the chromosome was performed by transforming the kanamycin-resistant strain with pCP20 an ampicillin-resistant plasmid (Cherepanov, and Wackernagel, supra)). Transformants were spread onto LB plates containing 100 μg/mL ampicillin. Plasmid pCP20 carries the yeast FLP recombinase under the control of the λ_{PR} promoter and expression from this promoter is controlled by the cl857 temperature-sensitive repressor residing on the plasmid. The origin of replication of pCP20 is also temperature-sensitive.

Removal of the IoxP-flanked kanamycin marker from the chromosome was performed by transforming the kanamycin-resistant strain with pJW168 an ampicillin-resistant plasmid (Wild et al., Gene. 223:55-66, 1998) harboring the bacteriophage P1 Cre recombinase. Cre recombinase (Hoess, R. H. & Abremski, K., supra) meditates excision of the kanamycin resistance gene via recombination at the IoxP sites. The origin of replication of pJW168 is the temperature-sensitive pSC101. Transformants were spread onto LB plates containing 100 μg/mL ampicillin.

Strain JW0886 (ΔpflB::kan) was transformed with plasmid pCP20 and spread on the LB plates containing 100 μg/mL ampicillin at 30° C. Ampicillin resistant transformants were then selected, streaked on the LB plates and grown at 42° C. Isolated colonies were patched onto the ampicillin and kanamycin selective medium plates and LB plates. Kanamycin-sensitive and ampicillin-sensitive colonies were screened by colony PCR with primers pflB CkUp (SEQ ID NO: 78) and pflB CkDn (SEQ ID NO: 79). A 10 μL aliquot of the PCR reaction mix was analyzed by gel electrophoresis. The expected approximate 0.4 kb PCR product was observed confirming removal of the marker and creating the “JW0886 markerless” strain. This strain has a deletion of the pflB gene.

The “JW0886 markerless” strain was transduced with a P1ᵥᵢᵣ lysate from JW4114 (frdB::kan) and streaked onto the LB plates containing 25 μg/mL kanamycin. The kanamycin-resistant transductants were screened by colony PCR with primers frdB CkUp (SEQ ID NO: 80) and frdB CkDn (SEQ ID NO: 81). Colonies that produced the expected approximate 1.6 kb PCR product were made electrocompetent and transformed with pCP20 for marker removal as described above. Transformants were first spread onto the LB plates containing 100 μg/mL ampicillin at 30° C. and ampicillin resistant transformants were then selected and streaked on LB plates and grown at 42° C. Isolated colonies were patched onto ampicillin and the kanamycin selective medium plates and LB plates. Kanamycin-sensitive, ampicillin-sensitive colonies were screened by PCR with primers frdB CkUp (SEQ ID NO: 80) and frdB CkDn (SEQ ID NO: 81). The expected approximate 0.4 kb PCR product was observed confirming marker removal and creating the double knockout strain, “ΔpflB frdB”.

The double knockout strain was transduced with a P1ᵥᵢᵣ lysate from JW1375 (ΔldhA::kan) and spread onto the LB plates containing 25 μg/mL kanamycin. The kanamycin-resistant transductants were screened by colony PCR with primers ldhA CkUp (SEQ ID NO: 82) and ldhA CkDn (SEQ ID NO: 83). Clones producing the expected 1.5 kb PCR product were made electrocompetent and transformed with pCP20 for marker removal as described above. Transformants were spread onto LB plates containing 100 μg/mL ampicillin at 30° C. and ampicillin resistant transformants were streaked on LB plates and grown at 42° C. Isolated colonies were patched onto ampicillin and kanamycin selective medium plates and LB plates. Kanamycin-sensitive, ampicillin-sensitive colonies were screened by PCR with primers ldhA CkUp (SEQ ID NO: 82) and ldhA CkDn (SEQ ID NO: 83) for a 0.3 kb product. Clones that produced the expected approximate 0.3 kb PCR product confirmed marker removal and created the triple knockout strain designated “3KO” (ΔpflB frdB ldhA).

Strain “3 KO” was transduced with a P1ᵥᵢᵣ lysate from JW1228 (ΔadhE::kan) and spread onto the LB plates containing 25 μg/mL kanamycin. The kanamycin-resistant transductants were screened by colony PCR with primers adhE CkUp (SEQ ID NO: 84) and adhE CkDn (SEQ ID NO: 85). Clones that produced the expected 1.6 kb PCR product were named 3KO adhE::kan. Strain 3KO adhE::kan was made electrocompetent and transformed with pCP20 for marker removal. Transformants were spread onto the LB plates containing 100 μg/mL ampicillin at 30° C. Ampicillin resistant transformants were streaked on the LB plates and grown at 42° C. Isolated colonies were patched onto ampicillin and kanamycin selective plates and LB plates. Kanamycin-sensitive, ampicillin-sensitive colonies were screened by PCR with the primers adhE CkUp (SEQ ID NO: 84) and adhE CkDn (SEQ ID NO: 85). Clones that produced the expected approximate 0.4 kb PCR product were named “4KO” (ΔpflB frdB ldhA adhE).

Construction of an E. coli Production Host (Strain NGCI-031) Containing an Isobutanol Biosynthetic Pathway And Deletions of pflB, frdB, ldhA, And adhE Genes

A DNA fragment encoding sadB, a butanol dehydrogenase, (DNA SEQ ID NO: 9; protein SEQ ID NO: 10) from Achromobacter xylosoxidans was amplified from A. xylosoxidans genomic DNA using standard conditions. The DNA was prepared using a Gentra Puregene kit (Gentra Systems, Inc., Minneapolis, Minn.; catalog number D-5500A) following the recommended protocol for gram negative organisms. PCR amplification was done using forward and reverse primers N473 and N469 (SEQ ID NOs: 86 and 87), respectively with Phusion High Fidelity DNA Polymerase (New England Biolabs, Beverly, Mass.). The PCR product was TOPO-Blunt cloned into pCR4 BLUNT (Invitrogen) to produce pCR4Blunt::sadB, which was transformed into E. coli Mach-1 cells. Plasmid was subsequently isolated from four clones, and the sequence verified.

The sadB coding region was then cloned into the vector pTrc99a (Amann et al., Gene 69: 301-315, 1988). The pCR4Blunt::sadB was digested with EcoRI, releasing the sadB fragment, which was ligated with EcoRI-digested pTrc99a to generate pTrc99a::sadB. This plasmid was transformed into E. coli Mach 1 cells and the resulting transformant was named Mach1/pTrc99a::sadB. The activity of the enzyme expressed from the sadB gene in these cells was determined to be 3.5 mmol/min/mg protein in cell-free extracts when analyzed using isobutyraldehyde as the standard.

The sadB gene was then subcloned into pTrc99A::budB-ilvC-ilvD-kivD as described below. The pTrc99A::budB-ilvC-ilvD-kivD is the pTrc-99a expression vector carrying an operon for isobutanol expression (described in Examples 9-14 the of U.S. Patent Application Publication No. 20070092957, which are incorporated herein by reference). The first gene in the pTrc99A::budB-ilvC-ilvD-kivD isobutanol operon is budB encoding acetolactate synthase from Klebsiella pneumoniae ATCC 25955, followed by the ilvC gene encoding acetohydroxy acid reductoisomerase from E. coli. This is followed by ilvD encoding acetohydroxy acid dehydratase from E. coli and lastly the kivD gene encoding the branched-chain keto acid decarboxylase from L. lactis.

The sadB coding region was amplified from pTrc99a::sadB using primers N695A (SEQ ID NO: 88) and N696A (SEQ ID NO: 89) with Phusion High Fidelity DNA Polymerase (New England Biolabs, Beverly, Mass.). Amplification was carried out with an initial denaturation at 98° C. for 1 min, followed by 30 cycles of denaturation at 98° C. for 10 sec, annealing at 62° C. for 30 sec, elongation at 72° C. for 20 sec and a final elongation cycle at 72° C. for 5 min, followed by a 4° C. hold. Primer N695A contained an AvrII restriction site for cloning and a RBS upstream of the ATG start codon of the sadB coding region. The N696A primer included an XbaI site for cloning. The 1.1 kb PCR product was digested with AvrII and XbaI (New England Biolabs, Beverly, Mass.) and gel purified using a Qiaquick Gel Extraction Kit (Qiagen Inc., Valencia, Calif.)). The purified fragment was ligated with pTrc99A::budB-ilvC-ilvD-kivD, that had been cut with the same restriction enzymes, using T4 DNA ligase (New England Biolabs, Beverly, Mass.). The ligation mixture was incubated at 16° C. overnight and then transformed into E. coli Mach 1™ competent cells (Invitrogen) according to the manufacturer's protocol. Transformants were obtained following growth on the LB agar with 100 μg/ml ampicillin. Plasmid DNA from the transformants was prepared with QIAprep Spin Miniprep Kit (Qiagen Inc., Valencia, Calif.) according to manufacturer's protocols. The resulting plasmid was called pTrc99A::budB-ilvC-ilvD-kivD-sadB.

Electrocompetent cells of the 4KO strains were prepared as described and transformed with pTrc99A::budB-ilvC-ilvD-kivD-sadB (“pBCDDB”). Transformants were streaked onto LB agar plates containing 100 μg/mL ampicillin. The resulting strain carrying plasmid pTrc99A::budB-ilvC-ilvD-kivD-sadB with 4KO was designated NGCI-031.

### Organic Extractants

The extractant is a water-immiscible organic solvent or solvent mixture having characteristics which render it useful for the extraction of butanol from a fermentation broth. A suitable organic extractant should meet the criteria for an ideal solvent for a commercial two-phase extractive fermentation for the production or recovery of butanol. Specifically, the extractant should (i) be biocompatible with the microorganisms, for example Escherichia coli, Lactobacillus plantarum, and Saccharomyces cerevisiae, (ii) be substantially immiscible with the fermentation medium, (iii) have a high partition coefficient (K_{P}) for the extraction of butanol, (iv) have a low partition coefficient for the extraction of nutrients, (v) have a low tendency to form emulsions with the fermentation medium, and (vi) be low cost and nonhazardous. In addition, for improved process operability and economics, the extractant should (vii) have low viscosity (μ), (viii) have a low density (ρ) relative to the aqueous fermentation medium, and (ix) have a boiling point suitable for downstream separation of the extractant and the butanol.

In one embodiment, the extractant may be biocompatible with the microorganism, that is, nontoxic to the microorganism or toxic only to such an extent that the microorganism is impaired to an acceptable level, so that the microorganism continues to produce the butanol product into the fermentation medium. The extent of biocompatibility of an extractant can be determined by the glucose utilization rate of the microorganism in the presence of the extractant and the butanol product, as measured under defined fermentation conditions. See, for example, the Examples in U.S. Provisional Patent Application Nos. 61/168,640; 61/168,642; and 61/168,645. While a biocompatible extractant permits the microorganism to utilize glucose, a non-biocompatible extractant does not permit the microorganism to utilize glucose at a rate greater than, for example, about 25% of the rate when the extractant is not present. As the presence of the fermentation product butanol can affect the sensitivity of the microorganism to the extractant, the fermentation product should be present during biocompatibility testing of the extractant. The presence of additional fermentation products, for example ethanol, may similarly affect the biocompatibility of the extractant. Use of a biocompatible extractant is desired for processes in which continued production of butanol is desired after contacting the fermentation broth comprising the microorganism with an organic extractant.

In one embodiment, the extractant may be selected from the group consisting of C₇ to C₂₂ fatty alcohols, C₇ to C₂₂ fatty acids, esters of C₇ to C₂₂ fatty acids, C₇ to C₂₂ fatty aldehydes, C₇ to C₂₂ fatty amides and mixtures thereof. Examples of suitable extractants include an extractant comprising at least one solvent selected from the group consisting of oleyl alcohol, behenyl alcohol, cetyl alcohol, lauryl alcohol, myristyl alcohol, stearyl alcohol, oleic acid, lauric acid, myristic acid, stearic acid, methyl myristate, methyl oleate, lauric aldehyde, 1-nonanol, 1-decanol, 1-undecanol, 2-undecanol, 1-nonanal, 2-butyloctanol, 2-butyl-octanoic acid and mixtures thereof. In embodiments, the extractant comprises oleyl alcohol. In embodiments, the extractant comprises a branched chain saturated alcohol, for example, 2-butyloctanol, commercially available as ISOFAL® 12 (Sasol, Houston, Tex.) or Jarcol I-12 (Jarchem Industries, Inc., Newark, N.J.). In embodiments, the extractant comprises a branched chain carboxylic acid, for example, 2-butyl-octanoic acid, 2-hexyl-decanoic acid, or 2-decyl-tetradecanoic acid, commercially available as ISOCARB® 12, ISOCARB® 16, and ISOCARB® 24, respectively (Sasol, Houston, Tex.).

In one embodiment, a first water-immiscible organic extractant may be selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof. Suitable first extractants may be further selected from the group consisting of oleyl alcohol, behenyl alcohol, cetyl alcohol, lauryl alcohol also referred to as 1-dodecanol, myristyl alcohol, stearyl alcohol, oleic acid, lauric acid, myristic acid, stearic acid, methyl myristate, methyl oleate, lauric aldehyde, and mixtures thereof. In one embodiment, the extractant may comprise oleyl alcohol.

In one embodiment, an optional second water-immiscible organic extractant may be selected from the group consisting of C₇ to C₂₂ fatty alcohols, C₇ to C₂₂ fatty carboxylic acids, esters of C₇ to C₂₂ fatty carboxylic acids, C₇ to C₂₂ fatty aldehydes, C₇ to C₂₂ fatty amides and mixtures thereof. Suitable second extractants may be further selected from the group consisting of 1-nonanol, 1-decanol, 1-undecanol, 2-undecanol, 1-nonanal, and mixtures thereof. In one embodiment, the second extractant comprises 1-decanol.

In one embodiment, the first extractant comprises oleyl alcohol and the second extractant comprises 1-decanol.

When a first and a second extractant are used, the relative amounts of each can vary within a suitable range. For example, the first extractant may be used in an amount which is about 30 percent to about 90 percent, or about 40 percent to about 80 percent, or about 45 percent to about 75 percent, or about 50 percent to about 70 percent of the combined volume of the first and the second extractants. The optimal range reflects maximization of the extractant characteristics, for example balancing a relatively high partition coefficient for butanol with an acceptable level of biocompatibility. For a two-phase extractive fermentation for the production or recovery of butanol, the temperature, contacting time, butanol concentration in the fermentation medium, relative amounts of extractant and fermentation medium, specific first and second extractants used, relative amounts of the first and second extractants, presence of other organic solutes including type and concentration of osmolytes, and the amount and type of microorganism are related; thus these variables may be adjusted as necessary within appropriate limits to optimize the extraction process as described herein.

Suitable organic extractants may be available commercially from various sources, such as Sigma-Aldrich (St. Louis, Mo.), in various grades, many of which may be suitable for use in extractive fermentation to produce or recover butanol. Technical grades of a solvent can contain a mixture of compounds, including the desired component and higher and lower molecular weight components. For example, one commercially available technical grade oleyl alcohol contains about 65% oleyl alcohol and a mixture of higher and lower fatty alcohols.

### Osmolyte

According to the present method, the fermentation medium contains at least one osmolyte at a concentration at least sufficient to increase the butanol partition coefficient relative to that in the presence of the osmolyte concentration of the basal fermentation medium and of an optional fermentable carbon source. The osmolyte may comprise one or more of the components of the basal fermentation medium, for example glucose, in which case the osmolyte is present at a concentration above that of the concentration of the osmolyte (e.g. glucose) contained in the basal fermentation medium. The osmolyte may comprise an optional fermentable carbon source present in the fermentation medium in addition to any fermentable carbon source included in the basal fermentation medium, for example xylose, in which case the osmolyte is present at a concentration above that of the optional fermentable carbon source in the fermentation medium. The osmolyte as defined in the definitions section above may comprise one or more organic substances which are not present in the basal fermentation medium or are not generally considered to be a fermentable carbon source, such as polyethylene glycol. The basal fermentation medium may contain a fermentable carbon source such as a monosaccharide and is generally tailored to a specific microorganism. Suggested compositions of basal fermentation media may be found in Difco™ & BBL™ manual (Becton Dickinson and Company, Sparks, Md. 21152, USA).

The osmolyte may comprise a monosaccharide, a disaccharide, glycerol, sugarcane juice, molasses, polyethylene glycol, dextran, high fructose corn syrup, corn mash, starch, cellulose, and combinations thereof. For example, the osmolyte may comprise a monosaccharide selected from the group consisting of glyceraldehyde, erythrose, threose, ribose, arabinose, xylose, lyxose, allose, altrose, glucose, mannose, gulose, idose, galactose, talose, dihydroxyacetone, erythrulose, ribulose, xylulose, psicose, fructose, sorbose, tagatose, and combinations thereof. For example, the osmolyte may comprise a disaccharide selected from the group consisting of sucrose, lactulose, lactose, maltose, trehalose, cellobiose, kojibiose, nigerose, isomaltose, sophorose, laminaribiose, gentiobiose, turanose, maltulose, palatinose, gentiobiulose, mannobiose, melibiose, melibiulose, rutinose, rutinulose, xylobiose, and combinations thereof. The osmolyte may be selected from the group consisting of polyethylene glycol, dextran, corn mash, starch, cellulose, and combinations thereof. Osmolytes selected from this group should be chosen to have molecular weight sufficiently high that they are not able to permeate into the microbial cell. A molecular weight of at least 8000 Daltons, for example, is desired for osmolytes selected from the group consisting of polyethylene glycol, dextran, corn mash, starch, cellulose, and combinations thereof.

The osmolyte may be available commercially from various sources in various grades, many of which may be suitable for use in extractive fermentation to produce or recover butanol by the methods disclosed herein. The osmolyte may be recovered by methods know in the art from a fermentation medium or from an aqueous phase formed by contacting the fermentation medium with an extractant or other physical or chemical methods such as precipitation, crystallization, and/or evaporation. The recovered osmolyte may be used in a subsequent fermentation. In one embodiment, the osmolyte may be obtained from a fermentation carbohydrate substrate, such as glucose from hydrolyzed corn mash, for example.

The amount of osmolyte needed to achieve a concentration in the fermentation medium at least sufficient to increase the butanol partition coefficient relative to that in the presence of the osmolyte concentration of the basal fermentation medium and of an optional fermentable carbon source can be determined as disclosed, for example, by the procedures of the Examples herein below. The range of osmolyte concentrations which have a positive effect on the partition coefficient is determined, for example by experimentation. The range of osmolyte concentrations which demonstrate acceptable biocompatibility with the microorganism of interest is also determined. The range of suitable osmolyte concentrations are then selected from the overlap of these two ranges, such that the amount of osmolyte required to have a positive effect on the butanol partition coefficient is balanced with the concentration range that provides an acceptable level of biocompatibility with the microorganism. Economic considerations may also be a factor in selecting the amount of osmolyte to use.

In one embodiment, the osmolyte may be present in the fermentation medium at a concentration which is biocompatible with the microorganism, that is, nontoxic to the microorganism or toxic only to such an extent that the microorganism is impaired to an acceptable level, so that the microorganism continues to produce the butanol product into the fermentation medium in the presence of the osmolyte. The extent of biocompatibility of an osmolyte can be determined by the growth rate of the microorganism in the presence of varying concentrations of the osmolyte. While a biocompatible osmolyte concentration permits the microorganism to utilize glucose, or another carbon source, or to grow, a non-biocompatible osmolyte concentration does not permit the microorganism to utilize glucose or another carbon source or to grow at a rate greater than, for example, about 25% of the growth rate when the excess amount of osmolyte is not present. The presence of fermentation products, for example butanol, may also affect the concentration ranges of the osmolyte which have biocompatibility with the microorganism. Use of an osmolyte within concentration ranges having biocompatibility is desired for processes in which continued production of butanol is necessary after contacting the fermentation medium comprising the microorganism with the osmolyte. In processes in which continued production of butanol after contacting the fermentation medium comprising the microorganism with the osmolyte is not required, an osmolyte may be used at concentration ranges which have little, if any, biocompatibility with the microorganism.

To achieve a concentration in the fermentation medium of osmolyte which is at least sufficient to increase the butanol partition coefficient relative to that in the presence of the osmolyte concentration of the basal fermentation medium and of an optional fermentable carbon source, the osmolyte may be added to the fermentation medium or to the aqueous phase of a biphasic fermentation medium during the growth phase of the microorganism, during the butanol production phase, when the butanol concentration is inhibitory, or to combinations thereof. The osmolyte may be added to the first extractant, to the second extractant, or to combinations thereof. The osmolyte may be added as a solid, as a slurry, or as an aqueous solution. Optionally, the osmolyte may be added to both the fermentation medium and the extractant(s). The osmolyte may be added in a continuous, semi-continuous, or batch manner. The osmolyte may be added to the entire stream to which it is introduced, for example to the entire fermentation medium in a fermentor, or to a partial stream taken from one or more vessels, for example to a partial stream taken from a fermentor.

In embodiments, the total concentration of osmolyte in the fermentation medium is at least about 0.2M, 0.3M, 0.4M, 0.5M, 0.6M, 0.7M, 0.8M, 0.9M, 1 M, or 2M. In some embodiments, the total concentration of osmolyte in the fermentation is less than about 5M.

### Fermentation

The microorganism may be cultured in a suitable fermentation medium in a suitable fermentor to produce butanol. Any suitable fermentor may be used including a stirred tank fermentor, an airlift fermentor, a bubble fermentor, or any combination thereof. Materials and methods for the maintenance and growth of microbial cultures are well known to those skilled in the art of microbiology or fermentation science (see for example, Bailey et al., Biochemical Engineering Fundamentals, second edition, McGraw Hill, New York, 1986). Consideration must be given to appropriate fermentation medium, pH, temperature, and requirements for aerobic, microaerobic, or anaerobic conditions, depending on the specific requirements of the microorganism, the fermentation, and the process. The fermentation medium used is not critical, but it must support growth of the microorganism used and promote the biosynthetic pathway necessary to produce the desired butanol product. A conventional fermentation medium may be used, including, but not limited to, complex media containing organic nitrogen sources such as yeast extract or peptone and at least one fermentable carbon source; minimal media; and defined media. Suitable fermentable carbon sources include, but are not limited to, monosaccharides, such as glucose or fructose; disaccharides, such as lactose or sucrose; oligosaccharides; polysaccharides, such as starch or cellulose; one carbon substrates; and mixtures thereof. In addition to the appropriate carbon source, the fermentation medium may contain a suitable nitrogen source, such as an ammonium salt, yeast extract or peptone, minerals, salts, cofactors, buffers and other components, known to those skilled in the art (Bailey et al., supra). Suitable conditions for the extractive fermentation depend on the particular microorganism used and may be readily determined by one skilled in the art using routine experimentation.

Methods for Recovering Butanol Using Extractive Fermentation with Added Osmolyte

Butanol may be recovered from a fermentation medium containing butanol, water, at least one osmolyte at a concentration at least sufficient to increase the butanol partition coefficient relative to that in the presence of the osmolyte concentration of the basal fermentation medium and of an optional fermentable carbon source, optionally at least one fermentable carbon source, and a microorganism that has been genetically modified (that is, genetically engineered) to produce butanol via a biosynthetic pathway from at least one carbon source. Such genetically modified microorganisms can be selected from bacteria, cyanobacteria, filamentous fungi and yeasts and include Escherichia coli, Lactobacillus plantarum, and Saccharomyces cerevisiae, for example. One step in the process is contacting the fermentation medium with a first water-immiscible organic extractant and optionally a second water-immiscible organic extractant to form a two-phase mixture comprising an aqueous phase and a butanol-containing organic phase. “Contacting” means the fermentation medium and the organic extractant or its solvent components are brought into physical contact at any time during the fermentation process. The osmolyte may be added to the fermentation medium, to the first extractant, to the optional second extractant, or to combinations thereof. In one embodiment, the fermentation medium further comprises ethanol, and the butanol-containing organic phase can contain ethanol.

When a first and a second extractant are used, the contacting may be performed with the first and second extractants having been previously combined. For example, the first and second extractants may be combined in a vessel such as a mixing tank, and the combined extractants may then be added to a vessel containing the fermentation medium. Alternatively, the contacting may be performed with the first and second extractants becoming combined during the contacting. For example, the first and second extractants may be added separately to a vessel which contains the fermentation medium. In one embodiment, contacting the fermentation medium with the organic extractant further comprises contacting the fermentation medium with the first extractant prior to contacting the fermentation medium and the first extractant with the second extractant. In one embodiment, the contacting with the second extractant may occur in the same vessel as the contacting with the first extractant. In one embodiment, the contacting with the second extractant may occur in a different vessel from the contacting with the first extractant. For example, the first extractant may be contacted with the fermentation medium in one vessel, and the contents transferred to another vessel in which contacting with the second extractant occurs. In these embodiments, the osmolyte may be added to the fermentation medium, to the first extractant, to the optional second extractant, or to combinations thereof.

The organic extractant may contact the fermentation medium at the start of the fermentation forming a biphasic fermentation medium. Alternatively, the organic extractant may contact the fermentation medium after the microorganism has achieved a desired amount of growth, which can be determined by measuring the optical density of the culture. In one embodiment, the first extractant may contact the fermentation medium in one vessel, and the second extractant may contact the fermentation medium and the first extractant in the same vessel. In another embodiment, the second extractant may contact the fermentation medium and the first extractant in a different vessel from that in which the first extractant contacts the fermentation medium. In these embodiments, the osmolyte may be added to the fermentation medium, to the first extractant, to the optional second extractant, or to combinations thereof.

Further, the organic extractant may contact the fermentation medium at a time at which the butanol level in the fermentation medium reaches a preselected level, for example, before the butanol concentration reaches a toxic or an inhibitory level. The butanol concentration may be monitored during the fermentation using methods known in the art, such as by gas chromatography or high performance liquid chromatography. The osmolyte may be added to the fermentation medium before or after the butanol concentration reaches a toxic or an inhibitory level. In embodiments, the organic extractant comprises fatty acids. In embodiments, processes described herein can be used in conjunction with processes described in U.S. Provisional Patent Application Nos. 61/368,429 and 61/379,546 wherein butanol is esterified with an organic acid such as fatty acid using a catalyst such as a lipase to form butanol esters.

Fermentation may be run under aerobic conditions for a time sufficient for the culture to achieve a preselected level of growth, as determined by optical density measurement. The osmolyte may be added to the fermentation broth before or after the preselected level of growth is achieved. An inducer may then be added to induce the expression of the butanol biosynthetic pathway in the modified microorganism, and fermentation conditions are switched to microaerobic or anaerobic conditions to stimulate butanol production, as described in detail in Example 6 of copending U.S. patent application Ser. No. 12/478,389. The extractant may be added after the switch to microaerobic or anaerobic conditions. The osmolyte may be added before or after the switch to microaerobic or anaerobic conditions. In one embodiment, the first extractant may contact the fermentation medium prior to the contacting of the fermentation medium and the first extractant with the second extractant. For example, in a batch fermentation process, a suitable period of time may be allowed to elapse between contacting the fermentation medium with the first and the second extractants. In a continuous fermentation process, contacting the fermentation medium with the first extractant may occur in one vessel, and contacting of that vessel's contents with the second extractant may occur in a second vessel. In these embodiments, the osmolyte may be added to the fermentation medium, to the first extractant, to the optional second extractant, or to combinations thereof.

After contacting the fermentation medium with the organic extractant in the presence of the osmolyte, the butanol product partitions into the organic extractant, decreasing the concentration in the aqueous phase containing the microorganism, thereby limiting the exposure of the production microorganism to the inhibitory butanol product. The volume of the organic extractant to be used depends on a number of factors, including the volume of the fermentation medium, the size of the fermentor, the partition coefficient of the extractant for the butanol product, the osmolyte concentration, and the fermentation mode chosen, as described below. The volume of the organic extractant may be about 3% to about 60% of the fermentor working volume. The ratio of the extractant to the fermentation medium is from about 1:20 to about 20:1 on a volume:volume basis, for example from about 1:15 to about 15:1, or from about 1:12 to about 12:1, or from about 1:10 to about 10:1, or from about 1:9 to about 9:1, or from about 1:8 to about 8:1.

The amount of the osmolyte to be added depends on a number of factors, including the effect of the added osmolyte on the growth properties of the butanol producing microorganism and the effect of the added osmolyte on the Kp of butanol in a two phase fermentation. The optimum amount of osmolyte to be added may also be dependent on the composition of the initial basal fermentation medium and the concentration of fermentable carbon source(s) in the fermentation medium. Too high a concentration of an osmolyte, although possibly increasing the Kp of butanol and alleviating the toxicity effects of butanol on the microorganism, can itself be inhibitory to the microorganism. On the other hand, too low a concentration of osmolyte might not increase the Kp of butanol sufficiently to alleviate the inhibitory effect of butanol on the microorganism. Therefore, a balance needs to be found through experimentation to ensure that the net effect of adding excess osmolyte to the fermentation medium results in an overall increase in the rate and titer of butanol production.

In embodiments, the Kp is increased by about 10%, about 20%, about 30%, about 40%, about 50%, about 60%, about 70%, about 80%, about 90%, about 100%, about 150%, or about 200% as compared to the Kp without added osmolyte. In embodiments, the Kp is increased by at least about 2-fold, at least about 3-fold, at least about 4 fold, at least about 5-fold, or at least about 6-fold. In embodiments, the total concentration of osmolyte is selected to increase the Kp by an amount while maintaining the growth rate of the microorganism at a level that is at least about 25%, at least about 50%, at least about 80%, or at least about 90% of the growth rate in the absence of added osmolyte. In embodiments, the total concentration of osmolyte in the fermentation medium is sufficient to increase the effective rate of butanol production by at least about 10%, at least about 20%, at least about 30%, at least about 40%, at least about 50%, at least about 60%, at least about 70%, at least about 80%, at least about 90%, or at least about 100% as compared to the rate without added osmolyte. In embodiments, the total concentration of osmolyte in the fermentation medium is sufficient to increase the effective yield of butanol by at least about 10%, at least about 20%, at least about 30%, at least about 40%, at least about 50%, at least about 60%, at least about 70%, at least about 80%, at least about 90%, or at least about 100% as compared to the effective yield without added osmolyte. In embodiments, the total concentration of osmolyte in the fermentation medium is sufficient to increase the effective titer of butanol by at least about 10%, at least about 20%, at least about 30%, at least about 40%, at least about 50%, at least about 60%, at least about 70%, at least about 80%, at least about 90%, or at least about 100% as compared to the effective titer without added osmolyte.

In embodiments, the amount of added osmolyte is sufficient to result in an effective titer of at least about 7 g/L, at least about 10 g/L, at least about 15 g/L, at least about 20 g/L, at least about 25 g/L, at least about 30 g/L, or at least about 40 g/L. In embodiments, the amount of added osmolyte is sufficient to result in an effective yield of at least about 0.12, at least about 0.15, at least about 0.2, at least about 0.25, or at least about 0.3. In embodiments, the amount of added osmolyte is sufficient to result in an effective rate of at least about 0.1 g/L/h, at least about 0.15 g/L/h, at least about 0.2 g/L/h, at least about 0.3 g/L/h, at least about 0.4 g/L/h or at least about 0.6 g/L/h, or at least about 0.8 g/L/h, or at least about 1 g/L/h or at least about 1.2 g/L/h. In some embodiments, the rate is about 1.3 g/L/h.

The next step is optionally separating the butanol-containing organic phase from the aqueous phase using methods known in the art, including but not limited to, siphoning, decantation, centrifugation, using a gravity settler, and membrane-assisted phase splitting. Recovery of the butanol from the butanol-containing organic phase may be done using methods known in the art, including but not limited to, distillation, adsorption by resins, separation by molecular sieves, and pervaporation. Specifically, distillation may be used to recover the butanol from the butanol-containing organic phase. The osmolyte may be recycled to the butanol production and/or recovery process.

The osmolyte may be recovered from the fermentation medium or from the aqueous phase of a two phase mixture by methods known in the art. For example, the aqueous phase or fermentation medium may be concentrated by distillation, stripping, pervaporation, or other methods to obtain a concentrated aqueous mixture comprising the osmolyte. Optionally, the osmolyte may be returned to a fermentation medium and thus be recycled within the fermentation process. Optionally, the osmolyte obtained from a fermentation carbohydrate substrate may be added to a fermentation medium to provide a concentration at least sufficient to increase the butanol partition coefficient relative to that in the presence of the osmolyte concentration of the basal fermentation medium and of an optional fermentable carbon source.

Gas stripping may be used concurrently with the organic extractant and the addition of osmolyte to remove the butanol product from the fermentation medium. Gas stripping may be done by passing a gas such as air, nitrogen, or carbon dioxide through the fermentation medium, thereby forming a butanol-containing gas phase. The butanol product may be recovered from the butanol-containing gas phase using methods known in the art, such as using a chilled water trap to condense the butanol, or scrubbing the gas phase with a solvent.

Any butanol remaining in the fermentation medium after the fermentation run is completed may be recovered by continued extraction using fresh or recycled organic extractant. Alternatively, the butanol can be recovered from the fermentation medium using methods known in the art, such as distillation, azeotropic distillation, liquid-liquid extraction, adsorption, gas stripping, membrane evaporation, pervaporation, and the like. In the case where the fermentation medium is not recycled to the process, additional osmolyte may be added to further increase the butanol partition coefficient and improve the efficiency of butanol recovery.

The two-phase extractive fermentation method may be carried out in a continuous mode in a stirred tank fermentor. In this mode, the mixture of the fermentation medium and the butanol-containing organic extractant is removed from the fermentor. The two phases are separated by means known in the art including, but not limited to, siphoning, decantation, centrifugation, using a gravity settler, membrane-assisted phase splitting, and the like, as described above. After separation, the fermentation medium and the osmolyte therein may be recycled to the fermentor or may be replaced with fresh medium, to which additional osmolyte is added. Then, the extractant is treated to recover the butanol product as described above. The extractant may then be recycled back into the fermentor for further extraction of the product. Alternatively, fresh extractant may be continuously added to the fermentor to replace the removed extractant. This continuous mode of operation offers several advantages. Because the product is continually removed from the reactor, a smaller volume of organic extractant is required enabling a larger volume of the fermentation medium to be used. This results in higher production yields. The volume of the organic extractant may be about 3% to about 50% of the fermentor working volume; 3% to about 20% of the fermentor working volume; or 3% to about 10% of the fermentor working volume. It is beneficial to use the smallest amount of extractant in the fermentor as possible to maximize the volume of the aqueous phase, and therefore, the amount of cells in the fermentor. The process may be operated in an entirely continuous mode in which the extractant is continuously recycled between the fermentor and a separation apparatus and the fermentation medium is continuously removed from the fermentor and replenished with fresh medium. In this entirely continuous mode, the butanol product is not allowed to reach the critical toxic concentration and fresh nutrients are continuously provided so that the fermentation may be carried out for long periods of time. The apparatus that may be used to carryout these modes of two-phase extractive fermentations are well known in the art. Examples are described, for example, by Kollerup et al. in U.S. Pat. No. 4,865,973.

Batchwise fermentation mode may also be used. Batch fermentation, which is well known in the art, is a closed system in which the composition of the fermentation medium is set at the beginning of the fermentation and is not subjected to artificial alterations during the process. In this mode, the desired amount of osmolyte and a volume of organic extractant are added to the fermentor and the extractant is not removed during the process. The organic extractant may be formed in the fermentor by separate addition of the first and the optional second extractants, or the first and second extractants may be combined to form the extractant prior to the addition of any extractant to the fermentor. The osmolyte may be added to the fermentation medium, to the first extractant, to the optional second extractant, or to combinations thereof. Although this fermentation mode is simpler than the continuous or the entirely continuous modes described above, it requires a larger volume of organic extractant to minimize the concentration of the inhibitory butanol product in the fermentation medium. Consequently, the volume of the fermentation medium is less and the amount of product produced is less than that obtained using the continuous mode. The volume of the organic extractant in the batchwise mode may be 20% to about 60% of the fermentor working volume; or 30% to about 60% of the fermentor working volume. It is beneficial to use the smallest volume of extractant in the fermentor as possible, for the reason described above.

Fed-batch fermentation mode may also be used. Fed-batch fermentation is a variation of the standard batch system, in which the nutrients, for example glucose, are added in increments during the fermentation. The amount and the rate of addition of the nutrient may be determined by routine experimentation. For example, the concentration of critical nutrients in the fermentation medium may be monitored during the fermentation. Alternatively, more easily measured factors such as pH, dissolved oxygen, and the partial pressure of waste gases, such as carbon dioxide, may be monitored. From these measured parameters, the rate of nutrient addition may be determined. The amount of organic extractant used and its methods of addition in this mode is the same as that used in the batchwise mode, described above. The amount of added osmolyte may be the same as in other fermentation modes.

Extraction of the product may be done downstream of the fermentor, rather than in situ. In this external mode, the extraction of the butanol product into the organic extractant is carried out on the fermentation medium removed from the fermentor. The osmolyte may be added to the fermentation medium removed from the fermentor. The amount of extractant used is about 20% to about 60% of the fermentor working volume; or 30% to about 60% of the fermentor working volume. The fermentation medium may be removed from the fermentor continuously or periodically, and the extraction of the butanol product by the organic extractant may be done with or without the removal of the cells from the fermentation medium. The cells may be removed from the fermentation medium by means known in the art including, but not limited to, filtration or centrifugation. The osmolyte may be added to the fermentation medium before or after removal of the cells. After separation of the fermentation medium from the extractant by means described above, the fermentation medium may be recycled into the fermentor, discarded, or treated for the removal of any remaining butanol product. Similarly, the isolated cells may also be recycled into the fermentor. After treatment to recover the butanol product, the extractant may be recycled for use in the extraction process. Alternatively, fresh extractant may be used. In this mode the extractant is not present in the fermentor, so the toxicity of the extractant is much less of a problem. If the cells are separated from the fermentation medium before contacting with the extractant, the problem of extractant toxicity may be further reduced. Furthermore, using this external mode there is less chance of forming an emulsion and evaporation of the extractant is minimized, alleviating environmental concerns.

Methods for Production of Butanol Using Extractive Fermentation with Added Osmolyte

An improved method for the production of butanol is provided, wherein a microorganism that has been genetically modified to produce butanol via a biosynthetic pathway from at least one fermentable carbon source is grown in a biphasic fermentation medium comprising an aqueous phase and i) a first water-immiscible organic extractant and optionally ii) a second water-immiscible organic extractant, and the biphasic fermentation medium further comprises at least one osmolyte at a concentration at least sufficient to increase the butanol partition coefficient relative to that in the presence of the osmolyte concentration of the basal fermentation medium and of an optional fermentable carbon source. Such genetically modified microorganisms can be selected from bacteria, cyanobacteria, filamentous fungi and yeasts and include Escherichia coli, Lactobacillus plantarum, and Saccharomyces cerevisiae, for example. The first water-immiscible organic extractant may be selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof, and the optional second water-immiscible organic extractant may be selected from the group consisting of C₇ to C₂₂ alcohols, C₇ to C₂₂ carboxylic acids, esters of C₇ to C₂₂ carboxylic acids, C₇ to C₂₂ aldehydes, C₇ to C₂₂ amides and mixtures thereof, wherein the biphasic fermentation medium comprises from about 10% to about 90% by volume of the organic extractant. Alternatively, the biphasic fermentation medium may comprise from about 3% to about 60% by volume of the organic extractant, or from about 15% to about 50%. The microorganism is grown in the biphasic fermentation medium for a time sufficient to extract butanol into the extractant to form a butanol-containing organic phase. The at least sufficient concentration of the osmolyte in the fermentation medium may be achieved by adding osmolyte to the aqueous phase during the growth phase of the microorganism, to the aqueous phase during the butanol production phase, to the aqueous phase when the butanol concentration in the aqueous phase is inhibitory, to the first extractant, to the second extractant, or to combinations thereof.

In one embodiment, the fermentation medium further comprises ethanol, and the butanol-containing organic phase can contain ethanol. The butanol-containing organic phase is then separated from the aqueous phase, as described above. Subsequently, the butanol is recovered from the butanol-containing organic phase, as described above.

Also provided is a method for the production of butanol wherein a microorganism that has been genetically modified to produce butanol via a biosynthetic pathway from at least one carbon source is grown in a fermentation medium wherein the microorganism produces the butanol into the fermentation medium to produce a butanol-containing fermentation medium. Such genetically modified microorganisms can be selected from bacteria, cyanobacteria, filamentous fungi and yeasts and include Escherichia coli, Lactobacillus plantarum, and Saccharomyces cerevisiae, for example. At least one osmolyte is added to the fermentation medium to provide the osmolyte at a concentration at least sufficient to increase the butanol partition coefficient relative to that in the presence of the osmolyte concentration of the basal fermentation medium and of an optional fermentable carbon source. In one embodiment, the osmolyte may be added to the fermentation medium when the microorganism growth phase slows. In one embodiment, the osmolyte may be added to the fermentation medium when the butanol production phase is complete. At least a portion of the butanol-containing fermentation medium is contacted with a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides and mixtures thereof, and optionally ii) a second water-immiscible organic extractant selected from the group consisting of C₇ to C₂₂ alcohols, C₇ to C₂₂-carboxylic acids, esters of C₇ to C₂₂ carboxylic acids, C₇ to C₂₂ aldehydes, C₇ to C₂₂ amides, and mixtures thereof, to form a two-phase mixture comprising an aqueous phase and a butanol-containing organic phase. The butanol-containing organic phase is then separated from the aqueous phase, as described above. Subsequently, the butanol is recovered from the butanol-containing organic phase, as described above. At least a portion of the aqueous phase is returned to the fermentation medium. In one embodiment, the fermentation medium further comprises ethanol, and the butanol-containing organic phase can contain ethanol.

Isobutanol may be produced by extractive fermentation with the use of a modified Escherichia coli strain in combination with an oleyl alcohol as the organic extractant, as disclosed in U.S. patent application Ser. No. 12/478,389. The method yields a higher effective titer for isobutanol (i.e., 37 g/L) compared to using conventional fermentation techniques (see Example 6 of U.S. patent application Ser. No. 12/478,389). For example, Atsumi et al. (Nature 451(3):86-90, 2008) report isobutanol titers up to 22 g/L using fermentation with an Escherichia coli that was genetically modified to contain an isobutanol biosynthetic pathway. The higher butanol titer obtained with the extractive fermentation method disclosed in U.S. patent application Ser. No. 12/478,389 results at least in part from the removal of the toxic butanol product from the fermentation medium, thereby keeping the level below that which is toxic to the microorganism. It is reasonable to assume that the present extractive fermentation method employing the use of at least one osmolyte at a concentration at least sufficient to increase the butanol partition coefficient relative to that in the presence of the osmolyte concentration of the basal fermentation medium and of an optional fermentable carbon source as defined herein would be used in a similar way and provide similar results.

Butanol produced by the methods disclosed herein may have an effective titer of greater than 22 g per liter of the fermentation medium. Alternatively, the butanol produced by methods disclosed may have an effective titer of at least 25 g per liter of the fermentation medium. Alternatively, the butanol produced by methods described herein may have an effective titer of at least 30 g per liter of the fermentation medium. Alternatively, the butanol produced by methods described herein may have an effective titer of at least 37 g per liter of the fermentation medium.

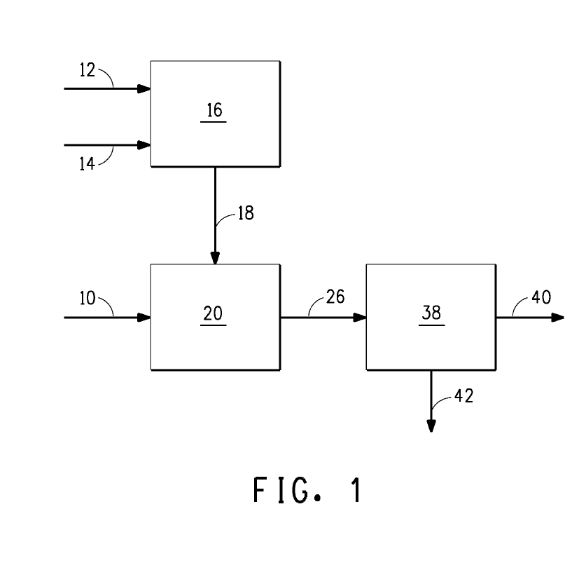

The present methods are generally described below with reference to FIG. 1 through FIG. 7.

Referring now to FIG. 1, there is shown a schematic representation of one embodiment of processes for producing and recovering butanol using in situ extractive fermentation. An aqueous stream 10 of at least one fermentable carbon source, optionally containing osmolyte, is introduced into a fermentor 20, which contains at least one genetically modified microorganism (not shown) that produces butanol from a fermentation medium comprising at least one fermentable carbon source. Optionally, osmolyte may be added as a separate stream (not shown) to the fermentor. A stream of the first extractant 12 and a stream of the optional second extractant 14 are introduced to a vessel 16, in which the first and second extractants are combined to form the combined extractant 18. Optionally, osmolyte may be added (not shown) to stream 18, to vessel 16, to the stream of the first extractant 12, to the stream of the second extractant 14, or to a combination thereof. A stream of the extractant 18 is introduced into the fermentor 20, in which contacting of the fermentation medium with the extractant to form a two-phase mixture comprising an aqueous phase and a butanol-containing organic phase occurs. A stream 26 comprising both the aqueous and organic phases is introduced into a vessel 38, in which separation of the aqueous and organic phases is performed to produce a butanol-containing organic phase 40 and an aqueous phase 42. Optionally, at least a portion of the aqueous phase 42 containing osmolyte is returned (not shown) to fermentor 20 or another fermentor (not shown). The point(s) of addition of the osmolyte to the process are selected such that the concentration of osmolyte in the aqueous phase 42 is at least sufficient to increase the butanol partition coefficient relative to that in the presence of the osmolyte concentration of the basal fermentation medium and of an optional fermentable carbon source.

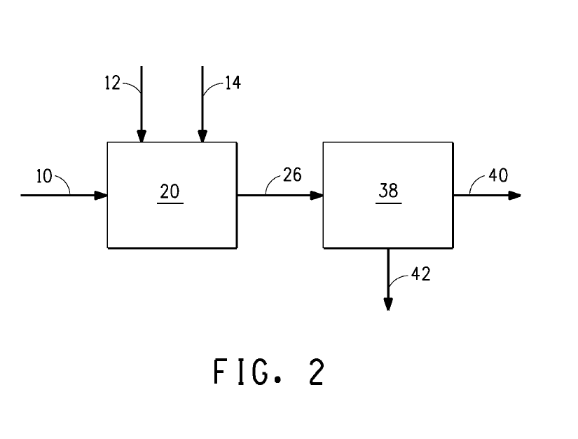

Referring now to FIG. 2, there is shown a schematic representation of one embodiment of processes for producing and recovering butanol using in situ extractive fermentation. An aqueous stream 10 of at least one fermentable carbon source, optionally containing osmolyte, is introduced into a fermentor 20, which contains at least one genetically modified microorganism (not shown) that produces butanol from a fermentation medium comprising at least one fermentable carbon source. Optionally, osmolyte may be added as a separate stream (not shown) to the fermentor. A stream of the first extractant 12 and a stream of the optional second extractant 14 are introduced separately to the fermentor 20, in which contacting of the fermentation medium with the extractant to form a two-phase mixture comprising an aqueous phase and a butanol-containing organic phase occurs. Optionally, osmolyte may be added (not shown) to stream 12, to stream 14, or to a combination thereof. A stream 26 comprising both the aqueous and organic phases is introduced into a vessel 38, in which separation of the aqueous and organic phases is performed to produce a butanol-containing organic phase 40 and an aqueous phase 42. Optionally, at least a portion of the aqueous phase 42 containing osmolyte is returned (not shown) to fermentor 20 or another fermentor (not shown). The point(s) of addition of the osmolyte to the process are selected such that the concentration of osmolyte in the aqueous phase 42 is at least sufficient to increase the butanol partition coefficient relative to that in the presence of the osmolyte concentration of the basal fermentation medium and of an optional fermentable carbon source.

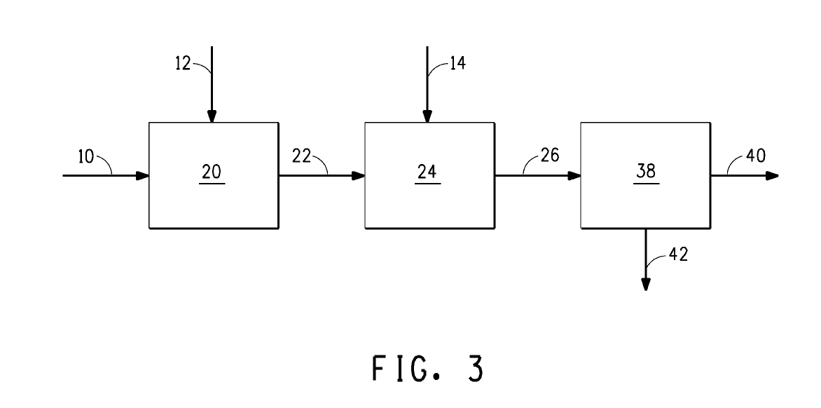

Referring now to FIG. 3, there is shown a schematic representation of one embodiment of processes for producing and recovering butanol using in situ extractive fermentation. An aqueous stream 10 of at least one fermentable carbon source, optionally containing osmolyte, is introduced into a first fermentor 20, which contains at least one genetically modified microorganism (not shown) that produces butanol from a fermentation medium comprising at least one fermentable carbon source. Optionally, osmolyte may be added as a separate stream (not shown) to the fermentor. A stream of the first extractant 12 is introduced to the fermentor 20, and a stream 22 comprising a mixture of the first extractant and the contents of fermentor 20 is introduced into a second fermentor 24. A stream of the optional second extractant 14 is introduced into the second fermentor 24, in which contacting of the fermentation medium with the extractant to form a two-phase mixture comprising an aqueous phase and a butanol-containing organic phase occurs. Optionally, osmolyte may be added (not shown) to stream 12, to stream 22, to stream 14, to vessel 24, or to a combination thereof. A stream 26 comprising both the aqueous and organic phases is introduced into a vessel 38, in which separation of the aqueous and organic phases is performed to produce a butanol-containing organic phase 40 and an aqueous phase 42. Optionally, at least a portion of the aqueous phase 42 containing osmolyte is returned (not shown) to fermentor 20 or another fermentor (not shown). The point(s) of addition of the osmolyte to the process are selected such that the concentration of osmolyte in the aqueous phase 42 is at least sufficient to increase the butanol partition coefficient relative to that in the presence of the osmolyte concentration of the basal fermentation medium and of an optional fermentable carbon source.

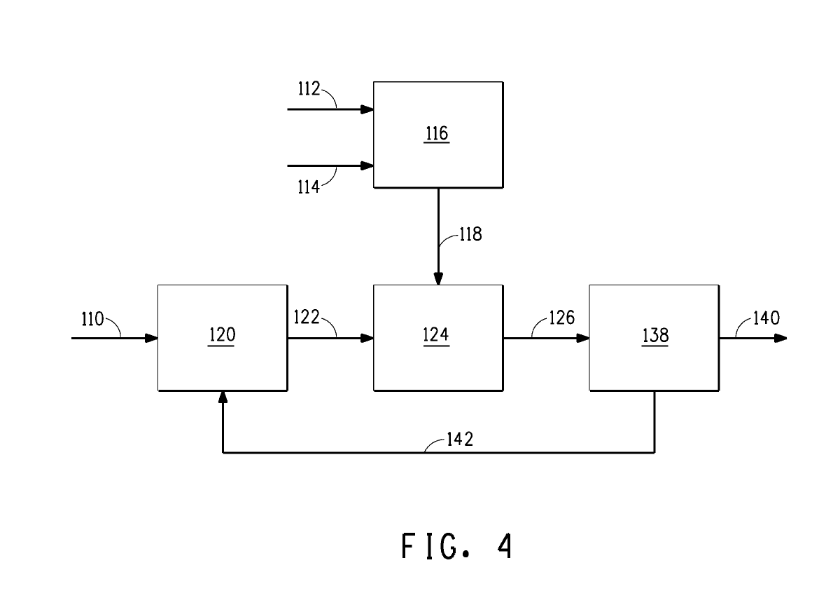

Referring now to FIG. 4, there is shown a schematic representation of one embodiment of processes for producing and recovering butanol in which extraction of the product is performed downstream of the fermentor, rather than in situ. An aqueous stream 110 of at least one fermentable carbon source, optionally containing osmolyte, is introduced into a fermentor 120, which contains at least one genetically modified microorganism (not shown) that produces butanol from a fermentation medium comprising at least one fermentable carbon source. Optionally, osmolyte may be added as a separate stream (not shown) to the fermentor. A stream of the first extractant 112 and a stream of the optional second extractant 114 are introduced to a vessel 116, in which the first and second extractants are combined to form the combined extractant 118. At least a portion, shown as stream 122, of the fermentation medium in fermentor 120 is introduced into vessel 124. Optionally, osmolyte may be added (not shown) to stream 112, to stream 114, to vessel 116, to stream 118, to vessel 124, or to a combination thereof. A stream of the extractant 118 is also introduced into vessel 124, in which contacting of the fermentation medium with the extractant to form a two-phase mixture comprising an aqueous phase and a butanol-containing organic phase occurs. A stream 126 comprising both the aqueous and organic phases is introduced into a vessel 138, in which separation of the aqueous and organic phases is performed to produce a butanol-containing organic phase 140 and an aqueous phase 142. At least a portion of the aqueous phase 142 containing osmolyte is returned to fermentor 120, or optionally to another fermentor (not shown). The point(s) of addition of the osmolyte to the process are selected such that the concentration of osmolyte in the aqueous phase 142 is at least sufficient to increase the butanol partition coefficient relative to that in the presence of the osmolyte concentration of the basal fermentation medium and of an optional fermentable carbon source.

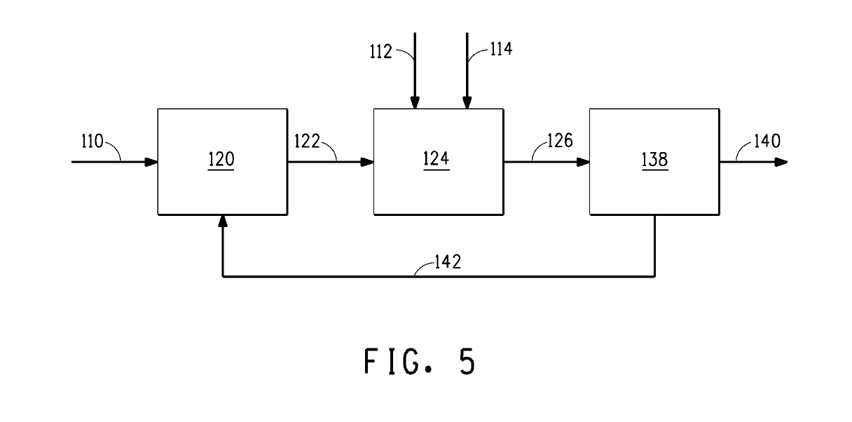

Referring now to FIG. 5, there is shown a schematic representation of one embodiment of processes for producing and recovering butanol in which extraction of the product is performed downstream of the fermentor, rather than in situ. An aqueous stream 110 of at least one fermentable carbon source, optionally containing osmolyte, is introduced into a fermentor 120, which contains at least one genetically modified microorganism (not shown) that produces butanol from a fermentation medium comprising at least one fermentable carbon source. Optionally, osmolyte may be added as a separate stream (not shown) to the fermentor. A stream of the first extractant 112 and a stream of the second extractant 114 are introduced separately to a vessel 124, in which the first and second extractants are combined to form the combined extractant. Optionally, osmolyte may be added (not shown) to stream 112, to stream 114, to stream 122, to vessel 124, or to combinations thereof. At least a portion, shown as stream 122, of the fermentation medium in fermentor 120 is also introduced into vessel 124, in which contacting of the fermentation medium with the extractant to form a two-phase mixture comprising an aqueous phase and a butanol-containing organic phase occurs. A stream 126 comprising both the aqueous and organic phases is introduced into a vessel 138, in which separation of the aqueous and organic phases is performed to produce a butanol-containing organic phase 140 and an aqueous phase 142. At least a portion of the aqueous phase 142 containing osmolyte is returned to fermentor 120, or optionally to another fermentor (not shown). The point(s) of addition of the osmolyte to the process are selected such that the concentration of osmolyte in the aqueous phase 142 is at least sufficient to increase the butanol partition coefficient relative to that in the presence of the osmolyte concentration of the basal fermentation medium and of an optional fermentable carbon source.

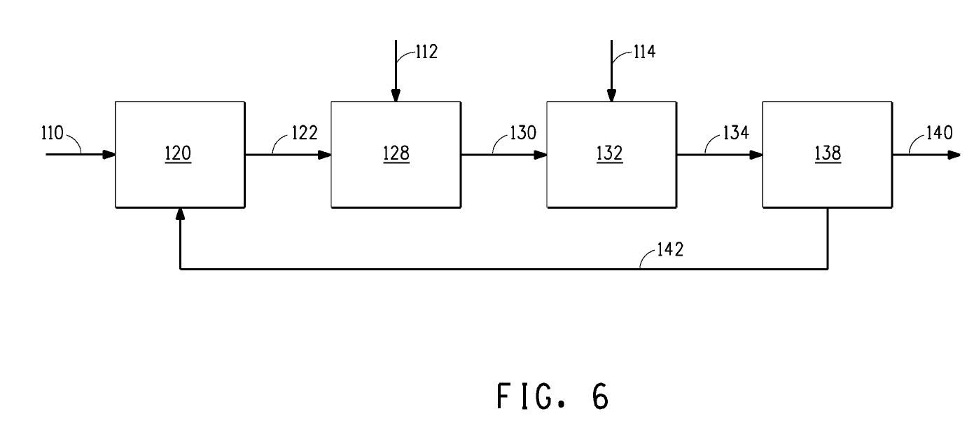

Referring now to FIG. 6, there is shown a schematic representation of one embodiment of processes for producing and recovering butanol in which extraction of the product is performed downstream of the fermentor, rather than in situ. An aqueous stream 110 of at least one fermentable carbon source, optionally containing osmolyte, is introduced into a fermentor 120, which contains at least one genetically modified microorganism (not shown) that produces butanol from a fermentation medium comprising at least one fermentable carbon source. Optionally, osmolyte may be added as a separate stream (not shown) to the fermentor. A stream of the first extractant 112 is introduced to a vessel 128, and at least a portion, shown as stream 122, of the fermentation medium in fermentor 120 is also introduced into vessel 128. Optionally, osmolyte may be added (not shown) to stream 122, to stream 112, to vessel 128, or to a combination thereof. A stream 130 comprising a mixture of the first extractant and the contents of fermentor 120 is introduced into a second vessel 132. Optionally, osmolyte may be added (not shown) to stream 130, to stream 114, to vessel 132, or to a combination thereof. A stream of the optional second extractant 114 is introduced into the second vessel 132, in which contacting of the fermentation medium with the extractant to form a two-phase mixture comprising an aqueous phase and a butanol-containing organic phase occurs. A stream 134 comprising both the aqueous and organic phases is introduced into a vessel 138, in which separation of the aqueous and organic phases is performed to produce a butanol-containing organic phase 140 and an aqueous phase 142. At least a portion of the aqueous phase 142 containing osmolyte is returned to fermentor 120, or optionally to another fermentor (not shown). The point(s) of addition of the osmolyte to the process are selected such that the concentration of osmolyte in the aqueous phase 142 is at least sufficient to increase the butanol partition coefficient relative to that in the presence of the osmolyte concentration of the basal fermentation medium and of an optional fermentable carbon source

The extractive processes described herein can be run as batch processes or can be run in a continuous mode where fresh extractant is added and used extractant is pumped out such that the amount of extractant in the fermentor remains constant during the entire fermentation process. Such continuous extraction of products and byproducts from the fermentation can increase effective rate, titer and yield.

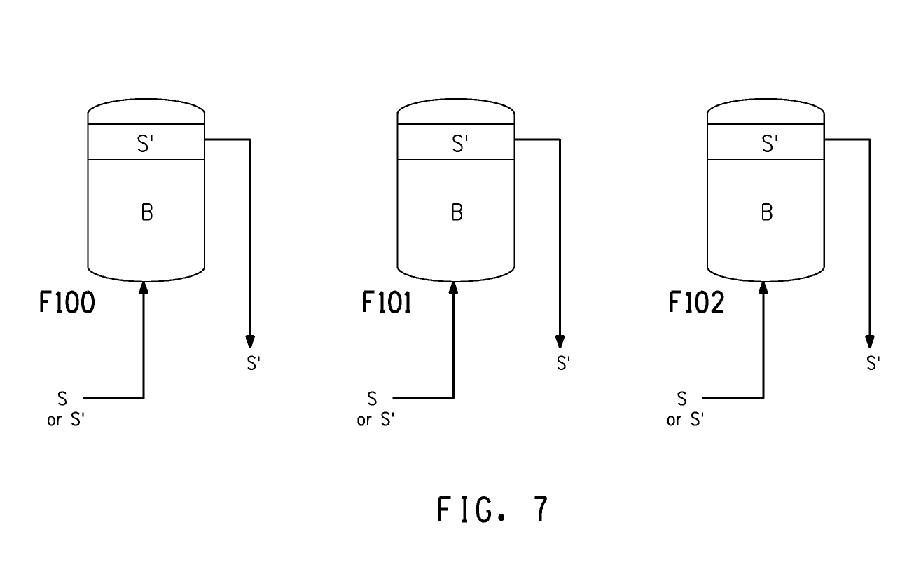

In yet another embodiment, it is also possible to operate the liquid-liquid extraction in a flexible co-current or, alternatively, counter-current way that accounts for the difference in batch operating profiles when a series of batch fermentors are used. In this scenario the fermentors are filled with fermentable mash which provides at least one fermentable carbon source and microorganism in a continuous fashion one after another for as long as the plant is operating. Referring to FIG. 7, once Fermentor F100 fills with mash and microorganism, the mash and microorganism feeds advance to Fermentor F101 and then to Fermentor F102 and then back to Fermentor F100 in a continuous loop. Osmolyte may be added (not shown) to one or more Fermentors, to the stream entering the Fermentor, to the stream exiting the fermentor, or a combination thereof. The fermentation in any one fermentor begins once mash and microorganism are present together and continues until the fermentation is complete. The mash and microorganism fill time equals the number of fermentors divided by the total cycle time (fill, ferment, empty and clean). If the total cycle time is 60 hours and there are 3 fermentors then the fill time is 20 hours. If the total cycle time is 60 hours and there are 4 fermentors then the fill time is 15 hours.

Adaptive co-current extraction follows the fermentation profile assuming the fermentor operating at the higher broth phase titer can utilize the extracting solvent stream richest in butanol concentration and the fermentor operating at the lowest broth phase titer will benefit from the extracting solvent stream leanest in butanol concentration. For example, referring again to FIG. 7, consider the case where Fermentor F100 is at the start of a fermentation and operating at relatively low butanol broth phase (B) titer, Fermentor F101 is in the middle of a fermentation operating at relatively moderate butanol broth phase titer and Fermentor F102 is near the end of a fermentation operating at relatively high butanol broth phase titer. In this case, lean extracting solvent (S), with minimal or no extracted butanol, can be fed to Fermentor F100, the “solvent out” stream (S′) from Fermentor F100 having an extracted butanol component can then be fed to Fermentor F101 as its “solvent in” stream and the solvent out stream from F101 can then be fed to Fermentor F102 as its solvent in stream. The solvent out stream from F102 can then be sent to be processed to recover the butanol present in the stream. The processed solvent stream from which most of the butanol is removed can be returned to the system as lean extracting solvent and would be the solvent in feed to Fermentor F100 above.

As the fermentations proceed in an orderly fashion the valves in the extracting solvent manifold can be repositioned to feed the leanest extracting solvent to the fermentor operating at the lowest butanol broth phase titer. For example, assume (a) Fermentor F102 completes its fermentation and has been reloaded and fermentation begins anew, (b) Fermentor F100 is in the middle of its fermentation operating at moderate butanol broth phase titer and (c) Fermentor F101 is near the end of its fermentation operating at relatively higher butanol broth phase titer. In this scenario the leanest extracting solvent would feed F102, the extracting solvent leaving F102 would feed Fermentor F100 and the extracting solvent leaving Fermentor F100 would feed Fermentor F101. The advantage of operating this way can be to maintain the broth phase butanol titer as low as possible for as long as possible to realize improvements in productivity. Additionally, it can be possible to drop the temperature in the other fermentors that have progressed further into fermentation that are operating at higher butanol broth phase titers. The drop in temperature can allow for improved tolerance to the higher butanol broth phase titers.

### Advantages of the Present Methods

The present extractive fermentation methods provide butanol known to have an energy content similar to that of gasoline and which can be blended with any fossil fuel. Butanol is favored as a fuel or fuel additive as it yields only CO₂ and little or no SO_{X} or NO_{X} when burned in the standard internal combustion engine. Additionally, butanol is less corrosive than ethanol, the most preferred fuel additive to date.

In addition to its utility as a biofuel or fuel additive, the butanol produced according to the present methods has the potential of impacting hydrogen distribution problems in the emerging fuel cell industry. Fuel cells today are plagued by safety concerns associated with hydrogen transport and distribution. Butanol can be easily reformed for its hydrogen content and can be distributed through existing gas stations in the purity required for either fuel cells or vehicles. Furthermore, the present methods produce butanol from plant derived carbon sources, avoiding the negative environmental impact associated with standard petrochemical processes for butanol production.

Advantages of the present methods include the feasibility of producing butanol at net effective rate, titer, and yield that are significantly higher and more economical than the threshold levels of butanol obtained by a two phase extractive fermentation process without the addition of at least one osmolyte at a concentration at least sufficient to increase the butanol partition coefficient relative to that in the presence of the osmolyte concentration of the basal fermentation medium and of an optional fermentable carbon source. The present method can also reduce the net amount of fresh or recycled extractant needed to achieve a desired level of butanol production from a batch fermentation.

### EXAMPLES

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

### Materials

The following materials were used in the examples. All commercial reagents were used as received.

All solvents were obtained from Sigma-Aldrich (St. Louis, Mo.) and were used without further purification. The oleyl alcohol used was technical grade, which contained a mixture of oleyl alcohol (65%) and higher and lower fatty alcohols. Isobutanol (purity 99.5%) was obtained from Sigma-Aldrich and was used without further purification.

### General Methods

Isobutanol and glucose concentrations in the aqueous phase were measured by HPLC (Waters Alliance Model, Milford, Mass. or Agilent 1200 Series, Santa Clara, Calif.) using a BioRad Aminex HPX-87H column, 7.8 mm×300 mm, (Bio-Rad laboratories, Hercules, Calif.) with appropriate guard columns, using 0.01 N aqueous sulfuric acid, isocratic, as the eluant. The sample was passed through a 0.2 μm centrifuge filter (Nanosep MF modified nylon) into an HPLC vial. The HPLC run conditions were as follows:

Injection volume: 10 μL

Flow rate: 0.60 mL/minute

Run time: 40 minutes

Column Temperature: 40° C.

Detector: refractive index

Detector temperature: 35° C.

UV detection: 210 nm, 8 nm bandwidth

After the run, concentrations in the sample were determined from standard curves for each of the compounds. The retention times were 32.6 and 9.1 minutes for isobutanol and glucose, respectively.

### Example 1

### Effect of Sucrose Concentration on the Partition Coefficient (Kₚ)

The purpose of this Example was to evaluate the effect of sucrose concentrations in the fermentation medium on the partition coefficient (Kₚ) of isobutanol when oleyl alcohol was used as the extractant. The basal fermentation medium (BFM) typically used in E. coli fermentations was used as the fermentation medium in this Example. The BFM composition is shown in Table 2.

TABLE 2
BFM Composition

| | Concentration (g/L) or as | Concentration |
|---|---|---|
| Components | indicated | (milli moles/L; mM) |
| Potassium phosphate | 13.3 | 97.73 |
| monobasic | | |
| Ammonium phosphate | 4.0 | 30.28 |
| dibasic | | |
| Citric acid monohydrate | 1.7 | 8.09 |
| Magnesium sulfate | 2.0 | 8.11 |
| heptahydrate | | |
| Trace Elements (mL/L) | 10.0 | — |
| Thiamine Hydrochloride | 4.5 | — |
| (mg/L) | | |
| Yeast Extract | 5.0 | — |
| Sigma Antifoam 204 | 0.20 | — |
| (mL/L) | | |
| Glucose | 30.0 | 170 |

The trace elements solution used in the above medium was prepared as follows. Ingredients listed below were added in the order listed and the solution is heated to 50° C.-60° C. until all the components are completely dissolved. Ferric citrate was added slowly after other ingredients were in solution. The solution was filter sterilized using 0.2 micron filters.

### EDTA 0.84 g/L

(Ethylenediaminetetraacetic acid
Cobalt dichloride hexahydrate 0.25 g/L
(cobalt chloride 6-hydrate)
Manganese dichloride tetrahydrate 1.5 g/L
(manganese chloride 4-hydrate)
Cupric chloride dihydrate 0.15 g/L
Boric acid (H₃BO₃) 0.30 g/L
Sodium molybdate dihydrate 0.25 g/L
Zinc acetate dihydrate 1.30 g/L
Ferric citrate 10.0 g/L

The initial level of total salts (sum of potassium phosphate monobasic, ammonium phosphate dibasic, citric acid monohydrate, and magnesium sulfate heptahydrate) in BFM as shown in Table 2 is calculated to be about 144.2 mM. Betaine Hydrochloride at 2 millimoles/L was added to the basal medium since it is well known in the literature (Cosquer A, et al; 1999; Appl Environ Microbiol 65:3304-3311) to improve osmotolerance tolerance of E. coli.

The following experimental procedure was used to generate the data in Table 3. In these Kₚ measurement experiments, a specified amount of sucrose was added as an osmolyte to the basal fermentation medium. To 30 mL of the sucrose-supplemented BFM, 10 mL of isobutanol rich oleyl alcohol (OA) extractant containing 168 g/L of isobutanol was added and mixed vigorously for 4 and 8 hours at 30° C. with shaking at 250 rpm in a table top shaker (Innova 4230, New Brunswick scientific, Edison, N.J.) to reach equilibrium between the two phases. The aqueous and organic phases in each flask were separated by decantation. The aqueous phase was centrifuged (2 minutes on 13,000 rpm with an Eppendorf centrifuge model 5415R) to remove residual extractant phase and the supernatant analyzed for glucose and isobutanol by HPLC. Analysis of isobutanol levels in the aqueous phase after 4 hrs of shaking was similar to that obtained following 8 hrs of mixing suggesting that equilibration between the two phases was attained within 4 hours. The intent was to prove that further mixing beyond 4 hrs did not change Kp.

Partition coefficients (Kₚ) for isobutanol distribution between the organic and aqueous phases were calculated from the known amount of isobutanol added to the flask and the isobutanol concentration data measured in the aqueous phase. The concentration of isobutanol in the extractant phase was determined by mass balance. The partition coefficient was determined as the ratio of isobutanol concentration in the organic and the aqueous phase, i.e., Kₚ=[Isobutanol]_{Organic phase}/[isobutanol]_{Aqueous phase}. Each data point corresponding to a specified level of sucrose as shown in Table 3 was repeated twice and values for Kₚ reported as the average of the two flasks.

TABLE 3
Effect of Sucrose Concentration on Kₚ of isobutanol

| Total initial | Amount of | Total | |
|---|---|---|---|
| concentration of | sucrose added | amount of | |
| sugars (Glucose) | to BFM | sugars in | |
| in BFM (Table 2) | Sucrose | experiment | |
| moles/L | (moles/L) | moles/L | |
| (a) | (b) | (a) + (b) | Kₚ |
| 0.17 | 0 | 0.17 | 4.35 |
| 0.17 | 0.03 | 0.20 | 4.44 |
| 0.17 | 0.09 | 0.26 | 4.41 |
| 0.17 | 0.17 | 0.34 | 4.60 |
| 0.17 | 0.26 | 0.43 | 4.69 |
| 0.17 | 0.33 | 0.50 | 5.09 |
| 0.17 | 0.51 | 0.68 | 5.21 |
| 0.17 | 0.67 | 0.84 | 5.85 |
| 0.17 | 1.00 | 1.17 | 6.85 |
| 0.17 | 1.33 | 1.50 | 7.77 |
| 0.17 | 2.00 | 2.17 | 10.69 |

Results from Table 3 demonstrate that supplementation of the aqueous fermentation medium with an osmolyte in the form of sucrose resulted in higher Kₚ for isobutanol in a two phase system with oleyl alcohol as the extractant phase.

### Example 2 (Prophetic)

### Increasing Isobutanol Production by Addition of an Excess Amount of Glucose or Sucrose as an Osmolyte in the Fermentation Medium

A genetically modified bacteria or yeast capable of producing isobutanol is grown in a typical fermentation medium that consists of some low levels of salts as a source of nitrogen and phosphate, vitamins, trace elements, yeast extract peptone, and a carbon source such as glucose or sucrose. The concentration of the carbon source typically varies from 2 g/L to 30 g/L. To encourage biomass production, the initial stage of the fermentation is aerobic in which air is sparged into the medium at 0.2-1.0 volume to volume per minute (vvm). Temperature is maintained at 30° C. and pH is maintained between 5.0 and 6.5. Once sufficient amount of biomass is grown, production of isobutanol is triggered by switching the fermentation to anaerobic conditions or microaerobic conditions. Anaerobic conditions are created by completely cutting off the air supply while microaerobic conditions are achieved by slowing down the supply of air and/or reducing the agitation speed. During this production stage of the fermentation, isobutanol accumulates in the medium and the concentration keeps building until it becomes inhibitory to the microorganism which results in slowing down of the fermentation rate. The net effect is lower overall rate and titer for isobutanol production.

Addition of organic extractants like oleyl alcohol into the fermentor during the production stage extracts butanol from the aqueous phase which alleviates its inhibitory effect on the microorganism resulting in higher rate and titer of isobutanol fermentation. Fermentation rate in this two phase system also slows down once the aqueous phase concentration of isobutanol reaches an inhibitory threshold level. In the presence of the extractant (oleyl alcohol) in the fermentor, the aqueous concentration of isobutanol is dictated by the partitioning coefficient (Kp) of isobutanol between the two phases. In the case of an oleyl alcohol/aqueous system, Kp is in the range of 3.5-4.5. A significant increase in isobutanol rate and titer can be achieved if Kp for isobutanol can be increased during fermentation such that the aqueous concentration of isobutanol drops below the inhibitory threshold level.

The results from Example 1 demonstrated that addition of high levels of sucrose can increase Kp dramatically, so once the aqueous concentration of isobutanol in Example 2 reaches inhibitory levels during fermentation, at least one osmolyte such as glucose, sucrose, corn mash, or combinations thereof is added to unusually high levels (50-250 g/L) to alleviate the inhibitory effect of the isobutanol on the microorganism. The net effect will be higher overall isobutanol fermentation rate and titer. Furthermore, the increase in Kp due to addition of such an osmolyte will lead to an improved and efficient extraction process during ISPR compared to the case in which no addition of excess sugars as osmolytes is made to the fermentation medium.

In one embodiment, the concentration of the osmolyte in the form of glucose can be modulated and varied during fermentation by controlling the rate of hydrolysis of the starch in corn mash to glucose. Corn mash, which predominantly comprises starch (polymer of glucose), is typically used as a source of carbon in the corn-to-ethanol industry to produce ethanol. In this process, the corn mash is first liquefied at high temperature (85° C.-100° C.) for 90-120 min by adding a thermostable alpha-amylase enzyme (for example SPEZYME® FRED-L; Genencor International, San Francisco, USA), then the liquefied corn mash is added to a fermentor containing an appropriate microorganism (biocatalyst) to produce either ethanol or butanol as described in this invention. The glucose in liquefied corn mash is slowly released during fermentation and made available to the microorganism by adding a second enzyme to the fermentor, for example glucoamylase (Distillase® L-400; Genencor International, San Francisco, USA). Typically, the rate of hydrolysis of starch which controls the rate of glucose availability in the fermentor is manipulated by the amount of glucoamylase enzyme added during fermentation. In this prophetic Example of butanol production, it is suggested that once butanol reaches an inhibitory level in the aqueous phase of the two-phase fermentor, the level of the osmolyte glucose can be increased to very high levels to maximize Kp of butanol by adding excess of glucoamylase. This method of modulating the level of glucose during butanol fermentation enables one to optimally deliver the osmolyte to both the growth phase and the production phase of the fermentation.

Analytical methods which could be used in prophetic Example 2 are described below.

Glucose concentration in the culture broth could be measured rapidly using a 2700 Select Biochemistry Analyzer (YSI Life Sciences, Yellow Springs, Ohio). Culture broth samples would be centrifuged at room temperature for 2 minutes at 13,200 rpm in 1.8 mL Eppendorf tubes, and the aqueous supernatant analyzed for glucose concentration. The analyzer could perform a self-calibration with a known glucose standard before assaying each set of fermentor samples; an external standard could also be assayed periodically to ensure the integrity of the culture broth assays. The analyzer specifications for the analysis could be as follows:

Sample size: 15 μL

Black probe chemistry: dextrose

White probe chemistry: dextrose

Isobutanol and ethanol in the organic extractant phase could be measured using Gas Chromatography (GC) as described below.

The following GC method could be used to determine the amount of isobutanol and ethanol in the organic phase. The GC method would utilize a J&W Scientific DB-WAXETR column (50 m×0.32 mm ID, 1 μm film) from Agilent Technologies (Santa Clara, Calif.). The carrier gas would be helium at a flow rate of 4 mL/min with constant head pressure; injector split would be 1:5 at 250° C.; oven temperature would be 40° C. for 5 min, 40° C. to 230° C. at 10° C./min, and 230° C. for 5 min. Flame ionization detection would be used at 250° C. with 40 mL/min helium makeup gas. Culture broth samples would be centrifuged before injection. The injection volume would 1.0 μL. Calibrated standard curves would be generated for ethanol and isobutanol. Under these conditions, the isobutanol retention time would be 9.9 minutes, and the retention time for ethanol would be 8.7 minutes.

Although particular embodiments of the present invention have been described in the foregoing description, it will be understood by those skilled in the art that the invention is capable of numerous modifications, substitutions, and rearrangements without departing from the spirit or essential attributes of the invention. Reference should be made to the appended claims, rather than to the foregoing specification, as indicating the scope of the invention.

What is claimed is:

1. A method for recovering butanol from a fermentation medium, the method comprising:
a) providing a fermentation medium comprising butanol, water, at least one osmolyte at a concentration at least sufficient to increase the butanol partition coefficient relative to that in the presence of the osmolyte concentration of the basal fermentation medium and of an optional fermentable carbon source, and a genetically modified microorganism that produces butanol from at least one fermentable carbon source;
b) contacting the fermentation medium with i) a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof, and optionally ii) a second water-immiscible organic extractant selected from the group consisting of C₇ to C₂₂ fatty alcohols, C₇ to C₂₂ fatty acids, esters of C₇ to C₂₂ fatty acids, C₇ to C₂₂ fatty aldehydes, C₇ to C₂₂ fatty amides and mixtures thereof to form a two-phase mixture comprising an aqueous phase and a butanol-containing organic phase; and
c) optionally, recovering the butanol from the butanol-containing organic phase to produce recovered butanol.

2. The method of claim 1, wherein a portion of the butanol is concurrently removed from the fermentation medium by a process comprising the steps of:
a) stripping butanol from the fermentation medium with a gas to form a butanol-containing gas phase; and
b) recovering butanol from the butanol-containing gas phase.

3. The method of claim 1, wherein the osmolyte is added to the fermentation medium, to the first extractant, to the optional second extractant, or to combinations thereof.

4. The method of claim 1, wherein the osmolyte comprises a monosaccharide, a disaccharide, glycerol, sugarcane juice, molasses, polyethylene glycol, dextran, high fructose corn syrup, corn mash, starch, cellulose, and combinations thereof.

5. The method of claim 1, wherein the osmolyte comprises a monosaccharide selected from the group consisting of sucrose, fructose, glucose, and combinations thereof.

6. The method of claim 1, wherein the osmolyte is selected from the group consisting of polyethylene glycol, dextran, corn mash, starch, cellulose, and combinations thereof.

7. The method of claim 1, wherein the genetically modified microorganism is selected from the group consisting of bacteria, cyanobacteria, filamentous fungi, and yeasts.

8. The method of claim 7 wherein the bacteria are selected from the group consisting of Zymomonas, Escherichia, Salmonella, Rhodococcus, Pseudomonas, Bacillus, Lactobacillus, Enterococcus, Pediococcus, Alcaligenes, Klebsiella, Paenibacillus, Arthrobacter, Corynebacterium, and Brevibacterium.

9. The method of claim 7 wherein the yeast is selected from the group consisting of Pichia, Candida, Hansenula, Kluyveromyces, Issatchenkia, and Saccharomyces.

10. The method of claim 1, wherein the first extractant is selected from the group consisting of oleyl alcohol, behenyl alcohol, cetyl alcohol, lauryl alcohol, myristyl alcohol, stearyl alcohol, oleic acid, lauric acid, myristic acid, stearic acid, methyl myristate, methyl oleate, lauric aldehyde, 1-dodecanol, and a combination of these.

11. The method of claim 1, wherein the first extractant comprises oleyl alcohol.

12. The method of claim 1, wherein the second extractant is selected from the group consisting of 1-nonanol, 1-decanol, 1-undecanol, 2-undecanol, 1-nonanal, and a combination of these.

13. The method of claim 1, wherein the butanol is 1-butanol.

14. The method of claim 1, wherein the butanol is 2-butanol.

15. The method of claim 1, wherein the butanol is isobutanol.

16. The method of claim 1, wherein the fermentation medium further comprises ethanol, and the butanol-containing organic phase contains ethanol.

17. The method of claim 1 wherein the genetically modified microorganism comprises a modification which inactivates a competing pathway for carbon flow.

18. The method of claim 1 wherein the genetically modified microorganism does not produce acetone.

19. A method for the production of butanol comprising:
a) providing a genetically modified microorganism that produces butanol from at least one fermentable carbon source;
b) growing the microorganism in a biphasic fermentation medium comprising an aqueous phase and i) a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides, and mixtures thereof, and optionally ii) a second water-immiscible organic extractant selected from the group consisting of C₇ to C₂₂ alcohols, C₇ to C₂₂-carboxylic acids, esters of C₇ to C₂₂ carboxylic acids, C₇ to C₂₂ aldehydes, C₇ to C₂₂ amides and mixtures thereof, wherein the biphasic fermentation medium further comprises at least one osmolyte at a concentration at least sufficient to increase the butanol partition coefficient relative to that in the presence of the osmolyte concentration of the basal fermentation medium and of an optional fermentable carbon source, for a time sufficient to allow extraction of the butanol into the organic extractant to form a butanol-containing organic phase;
c) separating the butanol-containing organic phase from the aqueous phase; and
d) optionally, recovering the butanol from the butanol-containing organic phase to produce recovered butanol.

20. The method of claim 19, wherein the osmolyte is added to the aqueous phase during the growth phase of the microorganism, to the aqueous phase during the butanol production phase, to the aqueous phase when the butanol concentration in the aqueous phase is inhibitory, to the first extractant, to the optional second extractant, or to combinations thereof.

21. The method of claim 20, wherein the osmolyte is obtained from a fermentation carbohydrate substrate.

22. A method for the production of butanol comprising:
a) providing a genetically modified microorganism that produces butanol from a fermentation medium comprising at least one fermentable carbon source;
b) growing the microorganism in a fermentation medium wherein the microorganism produces the butanol into the fermentation medium to produce a butanol-containing fermentation medium;
c) adding at least one osmolyte to the fermentation medium to provide the osmolyte at a concentration at least sufficient to increase the butanol partition coefficient relative to that in the presence of the osmolyte concentration of the basal fermentation medium and of an optional fermentable carbon source;
d) contacting at least a portion of the butanol-containing fermentation medium with i) a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides and mixtures thereof, and optionally ii) a second water-immiscible organic extractant selected from the group consisting of C₇ to C₂₂ alcohols, C₇ to C₂₂ carboxylic acids, esters of C₇ to C₂₂ carboxylic acids, C₇ to C₂₂ aldehydes, C₇ to C₂₂ amides and mixtures thereof, to form a two-phase mixture comprising an aqueous phase and a butanol-containing organic phase;
e) separating the butanol-containing organic phase from the aqueous phase;
f) optionally, recovering the butanol from the butanol-containing organic phase; and
g) optionally, returning at least a portion of the aqueous phase to the fermentation medium.

23. The method of claim 22, wherein the osmolyte is added to the fermentation medium in step (c) when the microorganism growth phase slows.

24. The method of claim 22, wherein the osmolyte is added to the fermentation medium in step (c) when the butanol production phase is complete.

25. The method of any one of claim 1, 19 or 22, wherein said at least one fermentable carbon source is present in the fermentation medium and comprises renewable carbon from agricultural feedstocks, algae, cellulose, hemicellulose, lignocellulose, or any combination thereof.

26. A composition comprising
(a) a fermentation medium comprising butanol, water, at least one osmolyte at a concentration at least sufficient to increase the butanol partition coefficient relative to that in the presence of the osmolyte concentration of the basal fermentation medium and of an optional fermentable carbon source, and a genetically modified microorganism that produces butanol from at least one fermentable carbon source;
b) a first water-immiscible organic extractant selected from the group consisting of C₁₂ to C₂₂ fatty alcohols, C₁₂ to C₂₂ fatty acids, esters of C₁₂ to C₂₂ fatty acids, C₁₂ to C₂₂ fatty aldehydes, C₁₂ to C₂₂ fatty amides and mixtures thereof; and
c) optionally a second water-immiscible organic extractant selected from the group consisting of C₇ to C₂₂ fatty alcohols, C₇ to C₂₂ fatty acids, esters of C₇ to C₂₂ fatty acids, C₇ to C₂₂ fatty aldehydes, C₁₂ to C₂₂ fatty amides and mixtures thereof;
wherein a two-phase mixture comprising an aqueous phase and a butanol-containing organic phase may form and whereby butanol may be separated from the fermentation medium of (a).
